(19) United States
(12) Patent Application Publication
(10) Pub. No.: US 2025/0263549 A1
(43) Pub. Date: Aug. 21, 2025

(54) THERMOPLASTIC ELASTOMER COMPOSITES, HYDROGEL COMPOSITES, AND GEL POLYMER ELECTROLYTE COMPOSITES

(71) Applicant: Colorado State University Research Foundation, Fort Collins, CO (US)

(72) Inventors: Travis S. Bailey, Fort Collins, CO (US); William B. Morris, Fort Collins, CO (US); Joseph Luther, Fort Collins, CO (US); Alyssa W. May, Fort Collins, CO (US); Justin Gangwish, Fort Collins, CO (US)

(73) Assignee: Colorado State University Research Foundation, Fort Collins, CO (US)

(21) Appl. No.: 18/857,348

(22) PCT Filed: Apr. 18, 2023
(86) PCT No.: PCT/US2023/018935

§ 371 Date: Oct. 16, 2024

Related U.S. Application Data
(60) Provisional application No. 63/363,148, filed on Apr. 18, 2022.

(51) Int. Cl. C08L 53/02; C08J 3/00; C08L 87/00

(52) U.S. Cl. CPC C08L 53/02; C08J 3/005; C08L 87/005; C08J 2353/02; C08J 2387/00; C08L 2207/04

(57) ABSTRACT

The present disclosure provides thermoplastic elastomer (TPE) composites, TPE hydrogel composites, TPE gel polymer electrolyte (GPE) composites, methods of preparing the TPE composites, methods of preparing the TPE hydrogel composites, methods of preparing the TPE GPE composites. The thermoplastic elastomer (TPE) composite comprises a first styrenic thermoplastic elastomer and a second styrenic thermoplastic elastomer, wherein the first and second styrenic thermoplastic elastomers each independently comprise at least one non-hydrogenated or hydrogenated styrene block.

### CROSS REFERENCE TO RELATED APPLICATIONS

This application claims priority to U.S. Provisional Patent Application Ser. No. 63/363,148, filed Apr. 18, 2022, the contents of which is hereby incorporated by reference for all purposes in its entirety.

### FIELD

The present disclosure relates to thermoplastic elastomer (TPE) composites, methods of preparing the TPE composites, TPE hydrogel composites, methods of preparing the TPE hydrogel composites, methods of using the TPE hydrogel composites, TPE gel polymer electrolyte (GPE) composites, methods of preparing the TPE GPE composites, TPE hydrogels and TPE GPEs. Aspects of the disclosure further relate to TPE hydrogels that exhibit mechanical toughness, fatigue resistance and fracture resistance with superior rates of elastic recovery and ionic conductivity comparable to the ionic conductivity of the liquid electrolyte medium.

### BACKGROUND

Low friction polymeric materials are used a large range of applications including, but not limited to pacemaker lead coatings, cardiac and urinary catheters, biomedical implant technologies, fabrics, fibers, filtration and separation membranes, and various electronic components. Many of these materials achieve their low friction surface characteristics from the integration of fluorine containing polymers or fluoropolymers such as ETFE or PTFE. However, a processing aid, used in the manufacture of these fluoropolymers materials is now being regulated, limiting the manufacture such low friction materials. PFAS (per and polyfluoroalkyl substances) are a human health and environmental concern due to the stability of these materials, sometimes referred to as ‘forever chemicals’ in the environment. The need for fluorine-free low friction polymer alternatives is thus becoming paramount.

Hydrogels and polymer electrolyte gels can be used in a large range of applications including, but not limited to, separation membranes, rapid ion-transport membranes, pharmaceuticals, biomedical materials, cosmetics, and personal hygiene products. Ideally, hydrogels and polymer electrolyte gels are tailorable to the performance demands of the intended application. Thus, hydrogels that are capable of sustaining repetitive stress loading without fatigue while suppressing susceptibility to fracture and failure are needed to ensure ideal performance. Effective integration of bulk toughness, durability and efficient elastic recovery, however, is limited using current hydrogel and polymer electrolyte gels design strategies. Accordingly, there is a need in the field for improved hydrogel and polymer electrolyte gels materials that can adequately handle demanding mechanical stresses.

Polymer composites are a class materials involving the mixing of more than one immiscible polymer to form a single material. Because the constituent polymers are immiscible and therefore thermodynamically unable to mix on the molecular scale, mechanical, thermal, or solvent assisted mixing is used to produce a microstructure maximizing the interfacial area of contact among the individual polymer domains with the hope of imparting the unique chemical, physical, or mechanical attributes of the constituent polymers to the resulting composite.

Historically, polymer composites formed in this manner suffer mechanically due to an inability to effectively pass mechanical energy or stress across the interfaces separating the constituent polymer domains. Strategies to improve energy transfer have included the use of block copolymers, typically diblock copolymers, which when located at the domain interfaces act as “compatibilizers” that help the transfer stress across the interface. However, such approaches have had limited success, because of the slow diffusion kinetics and limited ability to bring high molecular weight diblock copolymers to such interfaces effectively. Alternate strategies for compatibilizing the interface have been lacking.

This disclosure involves a strategy for forming mechanically durable and tough polymer composites, hydrogel composites, and gel polymer electrolyte composites by using a shared vitreous polymer domain to compatibilize the interface between constituent polymers.

### BRIEF DESCRIPTION OF THE DRAWINGS

Embodiments of the present inventive concept are illustrated by way of example in which like reference numerals indicate similar elements and in which:

FIG. 1A shows a two-liter anionic polymerization reactor with solvent delivery achieved through an inverted flask and monomer reactant delivered as a chilled liquid.

FIG. 1B shows one gram batch of SOS/SBS composite material recovered post-manufacture in a thermally processible powder form.

FIG. 2 depicts the ¹H NMR characterization data for the synthesized polystyrene-OH polystyrene-OH.

FIG. 3 depicts the ¹H NMR characterization data for the polystyrene-b-poly(ethylene oxide)-H, (SO—H).

FIG. 4 depicts the ¹H NMR characterization data for the “one-pot” polymerization of Polystyrene-b-poly(ethylene oxide)-b-polystyrene (SOS83).

FIG. 5 depicts the ¹H NMR characterization data for the “one-pot” polymerization of Polystyrene-b-poly(ethylene oxide)-b-polystyrene (SOS asw-2066).

FIG. 6 depicts the thermogravimetric analysis data for SOS asw-2066. Degradation occurring between 407.88° C. and 436.84° C.

FIG. 7 depicts the differential scanning calorimetry data for SOS asw-2066. The crystallization and glass transition of PEO is observed at about 70° C. The large presence of PEO covers what would be the glass transition of PS. However, it is possible that the sharp drop observed from 70° C. to 60° C. in the cooling cycle is the glass transition of PS.

FIG. 8 depicts the ¹H NMR characterization data for the Polystyrene-b-polybutadiene-b-polystyrene (SBS D-1102).

FIG. 9 depicts the thermogravimetric analysis data for SBS D-1102. Degradation occurring between 450.17° C. and 479.58° C.

FIG. 10 depicts the differential scanning calorimetry data for SBS D-1102. The glass transition of PB is observed at about −90° C. Due to the limitations of the instrument, the DSC can only measure to −90° C. Therefore, a large drop is seen right at the end of the cooling cycle.

FIG. 11 depicts one example of a TPE hydrogel composite production method using solvent blending.

FIG. 12 depicts thermogravimetric analysis data for the 50% SOS 50% SBS TPE hydrogel composite. Degradation of the SOS component occurs between 404.73° C. and about 450° C. which then transitions to the degradation of the SBS component, occurring between about 450° C. and 485.85° C. This transition can be seen in the inflection point around 450° C.

FIG. 13 depicts the differential scanning calorimetry data for the 50% SOS 50% SBS TPE hydrogel composite. The crystallization and glass transition of PEO is observed at about 70° C. The large presence of PEO covers what would be the glass transition of PS. The glass transition of PB is observed at about −90° C. Due to the limitations of the instrument, the DSC can only measure to −90° C. Therefore, a sharp drop is seen right at the end of the cooling cycle.

FIG. 14A, FIG. 14B, FIG. 14C, FIG. 14D, FIG. 14E, FIG. 14F, FIG. 14G depict SEM Images of freeze fractured surfaces of neat SOS hydrogel, solvent blended 50% SOS 50% SBS composite, and extruded 50% SOS 50% SEBS G-1650 composite. FIG. 14A depicts neat SOS hydrogel at ×1,000 magnification. FIG. 14B depicts neat SOS hydrogel at ×10,000 magnification.

FIG. 14C depicts solvent blended 50% SOS 50% SBS composite at ×1,000 magnification. FIG. 14D depicts solvent blended 50% SOS 50% SBS composite at ×10,000 magnification. FIG. 14E depicts solvent blended 50% SOS 50% SBS composite at ×10,000 magnification, SOS domains highlighted in blue, SBS domains highlighted in red to show the distinct domains after blending and thermal annealing of the composite. FIG. 14F depicts extruded 50% SOS 50% SEBS G-1650 composite at ×5,000 magnification. FIG. 14G depicts extruded 50% SOS 50% SEBS G-1650 composite at ×10,000 magnification. The data shows that the domain size of the TPE composite blend is smaller in the extruded 50% SOS 50% SEBS G-1650 composite compared to the 1 to 10 micrometer domain size of the solvent blended 50% SOS 50% SBS composite.

FIG. 15 depicts SAXS measurements of neat SOS hydrogel, neat SBS rubber, and solvent blended 50:50 SOS/SBS TPE composite all taken at 120° C. in situ, inside the Advanced Photon Source Synchrotron, after being annealed at 120° C. for 1 hour.

FIG. 16 depicts swollen and unswollen thermoplastic elastomer samples. Swollen samples are shown on the top row and the unswollen samples are shown on the bottom row of the sample image. Reading left to right by column the sample compositions are as follows: 100% neat SBS samples, 25% SOS 75% SBS TPE composite samples, 50% SOS 50% SBS TPE composite samples, 75% SOS 25% SBS TPE composite samples, 100% neat SOS samples.

FIG. 17 shows Tensile loading of a TPE hydrogel composite sample showing the sample in the unstretched (0% strain) and stretched state (1000% strain).

FIGS. 18A, 18B, and 18C show preliminary tensile (FIG. 18A and FIG. 18B) and compression data (FIG. 18C) on a series TPE hydrogel composites and their parent materials, blended by freeze drying in a cosolvent and processed using thermal melt pressing. Tension or compression applied at a rate of 2% strain per second. The data shows the modulus and toughness are enhanced as the concentration of the non-hydrogel elastomer in the TPE hydrogel composite increases, and the water content decreases. The concentration ratio between hydrogel elastomer and non-hydrogel elastomer can be varied in order to tune the mechanical properties and water absorption of the resulting composites.

FIG. 19A and FIG. 19B show preliminary tensile data of 25% SOS composites containing different non-swollen elastomer components. The composites were made with either SBS or one of two variants of SEBS. Composites made with A-1535 and G-1650 contain SEBS and the composite made with D-1102 contains SBS instead. The composites were blended with SOS by freeze drying in a cosolvent and processed using thermal melt pressing. Tension applied at a rate of 2% strain per second. The data shows that the 25% SOS 75% SEBS G-1650 composite exhibits the greatest young's modulus and toughness of the three samples. The young's modulus is enhanced in the 25% SOS 75% SEBS A-1535 compared to the 25% SOS 75% SBS D-1102 composite. However, both SEBS containing composites have lower elongation values compared to the SBS composite. Both the composites containing SEBS show strain hardening behavior but the composite containing SBS doesn't.

FIG. 20 depicts TPE hydrogel composite interface, figures listed left to right: 1 μm scale bar—component blend domains in a SOS/SEBS TPE hydrogel composite blend, 100 nm scale bar—block copolymer morphologies within component domains, 10 nm scale bar—microphase separation of block copolymer blocks, a with hypothesized demonstration of a shared vitreous polystyrene domain compatibilizing the TPE components at their interface.

FIG. 21A, FIG. 21B, FIG. 21C, and FIG. 21D show TPE hydrogel composite. FIG. 21A shows the TPE hydrogel composite thermally molded into the shape of an ovine medial meniscus at three sizes, each containing different water contents. FIG. 21B shows twisting action demonstrating the elastic nature of the implant under load. FIG. 21C shows an intact ovine medial meniscus. FIG. 21D shows a side by side comparison of an actual ovine medial meniscus and a prototype implant.

FIG. 21E shows the TPE hydrogel composite meniscal construct surgically implanted into the tibial plateau of a cadaver limb. All work done in collaboration with the CSU Preclinical Surgical Research Laboratory, CSU Veterinary School (Surgeon Jeremiah Easley).

FIG. 22 depicts the ¹H NMR characterization data for the 1-ethyl-3-methylimidazolium bromide as compared to the precursors.

FIG. 23 depicts the ¹H NMR characterization data for the 1-ethyl-3-methylimidazolium bis(trifluoromethylsulfonyl)imide.

FIG. 24A, FIG. 24B and FIG. 24C shows the ionic conductivities of the SOS ion gels (SOS83, SOS57, and SOS40), the neat ionic liquid, [EMIM][TFSI], and unswollen SOS83 discs which were measured using electrochemical impedance spectroscopy (EIS) while under vacuum.

FIG. 25 shows a graph of the ionic conductivities of the SOS ion gels (SOS83, SOS57, and SOS40), the neat ionic liquid, [EMIM][TFSI], and unswollen SOS83 discs the polymer was heated beyond the melting point of crystalline PEO domains after the polymer was heated beyond the melting point of crystalline PEO domains.

FIG. 26 shows a graph depicting the ionic conductivity of SOS gels, unswollen SOS, and RTIL as a function of temperature during a second thermal cycling starting at 80° C.

FIG. 27 shows a graph depicting a zoomed-in comparison of the ionic conductivity of the SOS gels and RTIL during the first and second heating cycles. Thermal annealing at higher temperatures does not significantly impact ionic conductivity of the gels.

FIG. 28A, FIG. 28B, FIG. 28C, and FIG. 28D shows the bulk mechanical properties of three blends of SOS GPE elastomers while under FIG. 28A tensile strain until failure, FIG. 28B cyclical compressive strain from 0 to 50% strain, and FIG. 28C oscillatory shear at varying frequencies with shear strains between 0.3 and 0.6%, depending on the linear viscoelastic regime of each gel (determined by dynamic strain sweeps). FIG. 28D shows a comparison of modulus extracted from parts a, b, and c.

FIGS. 29A-29C show graphical representations of the dynamic temperature ramps from 0 to 100° C. of the SOS ion gels.

FIGS. 30A and 30B depict the SOS40 gel under cyclical tensile loading at 10% strain s⁻¹. The gel exhibits no hysteresis, excellent elasticity, rapid recovery, and virtually no decay in modulus when repeatedly loaded. The failure at cycle 162 is likely due to initiation and propagation of a crack in the material due to a flaw from the melt processing step. The relatively high amplitude (200% strain) used in this test demonstrates the excellent flexibility and durability of the material in non-ideal conditions.

FIG. 31A shows the geometry of the “pure shear” fracture test.

FIG. 31B shows the force versus stretch data for the SOS40 control (black line) and the second SOS40 notched sample tested. It is highly unusual for a sample with an introduced crack to stretch beyond the failure point of the control, but we believe that the crack branching behavior, seen here as jagged “teeth” in the force-displacement plot, acts as a toughening mechanism.

FIG. 31C shows images and illustrated traces of notched sample 2 as it was stretched, and the crack propagated perpendicular to the initial crack.

FIG. 31D shows an unswollen disc of SOS40 after thermal processing.

FIG. 31E shows an SOS40 disc after swelling in RTIL to more than twice its initial size in all dimensions.

FIG. 31F shows a notched sample 2 after pure shear fracture testing. Note the tortuous crack path that is evidence of crack branching.

FIG. 32A, FIG. 32B, FIG. 32C, and FIG. 32D show the pure shear fracture testing data for an unnotched sample FIG. 32A and three notched samples (FIG. 326, FIG. 32C, and FIG. 32D) of SOS40. Evidence of the crack branching phenomenon can be seen as “dips” in the force-strain curve, where the crack propagated perpendicular to the path of the crack.

The drawing figures do not limit the present disclosure to the specific embodiments disclosed and described herein. The drawings are not necessarily to scale, emphasis instead being placed on clearly illustrating principles of certain embodiments of the present disclosure.

### SUMMARY

In some aspects, the current disclosure encompasses a thermoplastic elastomer (TPE) composite comprising a first styrenic thermoplastic elastomer and a second styrenic thermoplastic elastomer, wherein the first and second styrenic thermoplastic elastomers each independently comprise at least one non-hydrogenated or hydrogenated styrene block.

In some embodiments of the TPE composite, the first styrenic thermoplastic elastomer comprises at least one block copolymer comprising at least one non-styrenic hydrophilic block, and the second styrenic thermoplastic elastomers comprises at least one block copolymer comprising at least one non-styrenic hydrophobic block.

In some embodiments of the TPE composite, the first styrenic thermoplastic elastomer comprises at least one block copolymer comprising at least one non-styrenic hydrophilic block, and the second styrenic thermoplastic elastomer comprises at least one block copolymer comprising at least one non-styrenic hydrophilic block.

In some embodiments of the TPE composite, the first styrenic thermoplastic elastomer comprises at least one block copolymer comprising at least one non-styrenic hydrophobic block, and the second styrenic thermoplastic elastomer comprises at least one block copolymer comprising at least one non-styrenic hydrophobic block.

In some embodiments of the TPE composite as disclosed herein, each of the first styrenic thermoplastic elastomer or the second styrenic thermoplastic elastomer or both comprises diblock copolymers, triblock copolymers, tetrablock copolymers, pentablock copolymers, or any combination thereof. In some embodiments, the at least one hydrophilic block comprises at least one polyalkylene oxide block. In some embodiments, the at least one polyalkylene oxide block comprises a polyethylene oxide block. In some embodiments, the at least one hydrophobic block comprises at least one hydrophobic non-glassy block, non-limiting examples of which include a polydiene, a substituted polydiene, a hydrogenated polydiene, a substituted hydrogenated polydiene, a polysiloxane, a substituted polysiloxane, or any combination thereof.

In some embodiments, the ratio of the first styrenic thermoplastic elastomer to the second styrenic thermoplastic elastomers is 1:19 to 19:1. In some embodiments, the ratio of the first styrenic thermoplastic elastomer to the second styrenic thermoplastic elastomers is 1:4 to 4:1.

In some embodiments of the TPE composite as disclosed herein, the first styrenic thermoplastic elastomer or the second styrenic elastomer or both, comprising at least one non-styrenic hydrophilic block comprises a polystyrene-polyethylene oxide-polystyrene triblock copolymer (SOS). In some embodiments, the first styrenic thermoplastic elastomer or second styrenic thermoplastic elastomer or both, comprising at least one non-styrenic hydrophobic block comprises at least one non-styrenic hydrophobic block, non-limiting examples of which include a polystyrene-polybutadiene-polystyrene triblock copolymer (SBS), a substituted SBS triblock copolymer, a polystyrene-polyisoprene-polystyrene triblock copolymer (SIS), a substituted SIS triblock copolymer, a polystyrene-poly(ethylene butylene)-polystyrene triblock copolymer (SEBS), a substituted SEBS triblock copolymer, a polycyclohexylethylene-poly(ethylene butylene)-polycyclohexylethylene triblock copolymer (PEBP), a substituted PEBP triblock copolymer, a polystyrene-polysiloxane-polystyrene triblock copolymer (SDS), a substituted SDS triblock copolymer, or any combination thereof. In some embodiments of the TPE composite as disclosed herein, the at least two styrenic thermoplastic elastomers comprise SOS and SEBS. In some embodiments, the ratio of SOS to SEBS is 1:19 to 19:1.

In some aspects, the current disclosure also encompasses a method for preparing a thermoplastic elastomer (TPE) composite, comprising: (a) contacting the at least two thermoplastic elastomers as disclosed herein to form a TPE dry blend; (b) heating the TPE dry blend to form a TPE composite melt; and (c) allowing the TPE composite melt to attain ambient temperature to form an TPE composite of the at least two thermoplastic elastomers. In some embodiments, the TPE dry blend is formed by dissolving the at least two thermoplastic elastomers in a solvent and removing the solvent. In some embodiments, the TPE dry blend is formed by heating the at least two thermoplastic elastomers between about 80° C. and 320° C. In some embodiments, the TPE dry blend is formed by heating the at least two thermoplastic elastomers between about 5 minutes and 60 minutes. In some embodiments, the TPE dry blend is heated in the presence of applied pressure between about 1000 lbf and 25000 lbf. In some embodiments of the method as disclosed herein, the TPE dry blend has a microstructure characterized by TPE domains of sizes of about 0.1 microns to about 10 microns, about 0.1 microns to about 20 microns, or about 0.1 microns to about 50 microns.

In some embodiments of the method as disclosed herein, the step (b), heating the TPE dry blend, further comprises mechanical mixing, compounding, or extrusion. In some embodiments of the method as disclosed herein, the step (b), heating the TPE dry blend, comprises mechanical mixing with a screw or mixing speed between about 50 rpm and 250 rpm.

In some aspects, the current disclosure also encompasses a thermoplastic elastomer (TPE) hydrogel composite comprising: (a) any one of the thermoplastic elastomer (TPE) composites as disclosed herein; and (b) a liquid medium comprising one or more aqueous solvents, aqueous liquid electrolytes, or a combination thereof. In some embodiments, the liquid medium comprises water. In some embodiments, the liquid medium comprises an aqueous solvent or aqueous electrolyte. In some embodiments, the TPE hydrogel composite of as disclosed herein, has a water content of about 0.01% w/v to about 95% w/v. In some embodiments, the TPE hydrogel composite of as disclosed herein, has a bulk modulus averaged over the initial 10% strain of about 0.1 MPa to about 25 MPa. In some embodiments, the TPE hydrogel composite of as disclosed herein, has a toughness of about 1 MJ/m³ to about 120 MJ/m³. In some embodiments, the TPE hydrogel comprises a lubricious surface. In some embodiments, the TPE hydrogel is resistant to biofouling, fatigue, wear, fracture, degradation, or any combination thereof.

In some aspects, the current disclosure also encompasses a synthetic fibrocartilage mimic, such as a meniscus or intervertebral disc comprising a TPE hydrogel as disclosed herein. In some aspects, the current disclosure also encompasses a wound dressing comprising a TPE hydrogel composite as disclosed herein. In some aspects, the current disclosure also encompasses a catheter or catheter surface comprising a TPE hydrogel as disclosed herein. In some aspects, the current disclosure also encompasses a cardiac electrical lead comprising a TPE hydrogel composite as disclosed herein. In some aspects, the current disclosure also encompasses a coating applied to the surface of a biomedical device comprising a TPE hydrogel as disclosed herein.

In some aspects, the current disclosure also encompasses a method for preparing a thermoplastic elastomer (TPE) hydrogel composite, comprising: (a) obtaining a TPE composite as disclosed herein or a TPE composited formed by any one of the methods disclosed herein; and (b) contacting the TPE composite with a liquid medium to form the TPE hydrogel composite.

In some embodiments, the step (b) comprises contacting the TPE composite with the liquid medium occurs at a temperature above −10° C. and below about 70° C. In some embodiments, the TPE hydrogel composite has a liquid medium concentration between about 0.01 w/v % to about 95 w/v % liquid medium/TPE composite. In some embodiments, the liquid medium is chosen from an aqueous medium comprising at least one aqueous solvent or aqueous electrolyte.

In some aspects, the current disclosure also encompasses a thermoplastic elastomer (TPE) gel polymer electrolyte (GPE) composite comprising: (a) any one of the thermoplastic elastomer (TPE) composites as disclosed herein; and (b) a liquid medium comprising one or more non-aqueous solvents, or non-aqueous liquid electrolytes, or a combination thereof. In some embodiments of the TPE GPE composite as disclosed herein, the liquid medium comprises one or more room-temperature ionic liquids (RTIL), non-limiting examples of which include 1-ethyl-3-methyl imidazolium bis(trifluoromethane)sulfonamide ([EMIM][TFSI]), 1-hexyl-3-methyl imidazolium bis(trifluoromethane)sulfonamide ([HMIM][TFSI]), 1-vinyl-3-ethyl-imidazolium bis(trifluoromethane)sulfonamide ([VEIM][TFSI]), 1-allyl-3-methyl-imidazolium bis(trifluoromethane)sulfonamide ([AMIM][TFSI]), 1-hexyl-3-butyl-imidazolium bis(trifluoromethane)sulfonamide ([HBIM][TFSI]), 1-vinyl-3-methylimidazolium bis(trifluoromethane)sulfonamide ([VMIM][TFSI]), 1-hydroxyundecanyl-3-methylimidazolium bis(trifluoromethane)sulfonamide ([(C₁₁OH)MIM][TFSI]), 1-ethyl-3-methylimidazolium tricyanomethanide ([EMIM][TCM]), tetrabutylphosphonium taurinate, ([P4444][Tau]), 1-ethyl-3-methylimidazolium dicyanamide ([EMIM][DCA]), 1-(2,3-dihydroxypropyl)-alkyl imidazolium dicyanamide ([(dhp)MIM][DCA]), 1-(2,3-dihydroxypropyl)-3-alkyl imidazolium tetrafluoroborate ([(dhp)MIM][BF4]), 1-(2,3-dihydroxypropyl)-3-alkyl imidazolium bis(trifluoromethane)sulfonimide ([(dhp)MIM][TFSI]), and 1-(2,3-dihydroxypropyl)-3-alkyl imidazolium hexafluorophosphate ([(dhp)MIM][PF6]).

In some embodiments, the RTIL of the TPE GPE composite as disclosed herein of comprises an imidazolium RTIL. In some embodiments, the RTIL comprises 1-ethyl-3-methylimidazolium bis(trifluoromethanesulfonyl)imide. In some embodiments, the TPE GPE composite as disclosed herein has a non-aqueous solvent or non-aqueous liquid electrolyte content of about 0.01% w/v to about 95% w/v.

In some embodiments, the TPE GPE composite as disclosed herein has a bulk modulus averaged over the initial 10% strain of about 0.1 MPa to about 25 MPa. In some embodiments, the TPE GPE composite as disclosed herein has a toughness of about 1 MJ/m³ to about 120 MJ/m³. In some embodiments, the TPE GPE composite as disclosed herein has an ionic conductivity comparable to the ionic conductivity of the liquid electrolyte medium. In some embodiments, the TPE GPE composite is resistant to fatigue, wear, fracture, degradation, or any combination thereof. In some aspects, the current disclosure also encompasses a membrane, comprising a TPE gel polymer electrolyte composite as disclosed herein, wherein the membrane has a CO₂/N₂ selectivity between about 10:1 and about 60:1. In some aspects, the current disclosure also encompasses a battery separator, comprising a TPE gel polymer electrolyte composite as disclosed herein, wherein the battery separator has an ionic conductivity comparable to the ionic conductivity of the liquid electrolyte.

In some aspects, the current disclosure also encompasses a method for preparing a thermoplastic elastomer (TPE) gel electrolyte composite (GPE), comprising: (a) obtaining a TPE composite as disclosed herein, or a TPE composite formed by any one of the methods disclosed herein; and (b) contacting the TPE composite with a liquid medium to form the TPE GRE composite. In some embodiments, the step (b), contacting the TPE composite with the liquid medium, occurs at a temperature above −10° C. and below about 70° C. In some embodiments, the TPE hydrogel composite has a liquid medium concentration between about 0.01 w/v % to about 95 w/v % liquid medium/TPE composite. In some embodiments, the liquid medium is chosen from a non-aqueous medium comprising at least one non-aqueous solvent or non-aqueous electrolyte.

Other features and iterations of the disclosure are described in more detail below.

### DETAILED DESCRIPTION

The present disclosure may be understood by reference to the following detailed description, taken in conjunction with the drawings as described above.

The disclosure provides mechanically durable thermoplastic elastomer composites and hydrogels and gel polymer electrolytes formed from those composites. The disclosure is predicated on a new strategy for compatibilizing immiscible polymer blends and achieving effective mechanical stress transfer across the interface separating individual polymer domains. This new approach allows such composites to achieve bulk toughness values over 100 MJ/m³ and eliminates interfacial delamination or failure among the constituent domains as the primary mechanism for fracture.

Thermoplastic elastomer materials are known for utilizing microphase separation of ABA block copolymer blends to create reversible physical crosslinking. In this disclosure, the phase separation behavior is exploited to create super-tough elastomer composites by combining two or more thermoplastic elastomer materials which share a common vitreous polymer block. These composites are formed through the addition of thermal energy, mechanical energy, solvents or any combination of these to a dry blend of the two elastomers, followed by cooling or drying to form a solid composite blend. These composites have been found to exhibit bulk toughness in the 25-100 MJ/m³ ranges, reaching strains beyond 1500%. The two materials each form their own elastomeric network but use a shared vitreous component to maximize interfacial strength across the component domains.

This approach, in which two elastomers to be blended are synthesized to possess a common vitreous polymer block, allows the composite to generate a shared domain at all interfaces between the blended elastomers, providing the final solid composite with exceptional strength and toughness while still offering the chemical and physical properties inherent to the original elastomers.

In the case of a forming a thermoplastic elastomer hydrogel composite or a thermoplastic elastomer gel polymer electrolyte composite, one of the thermoplastic elastomers is designed to contain a hydrophilic or electrolyte soluble block, which provides the composite the ability to uptake water, aqueous solutions, non-aqueous solutions or solvents, and ionic liquids at levels up to 95%, while retaining the strength of an industrial grade elastomer.

### I. Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used herein, the terms “about” and “approximately” designate that a value is within a statistically meaningful range. Such a range can be typically within 20%, more typically within 10%, and even more typically within 5% of a given value or range. The allowable variation encompassed by the terms “about” and “approximately” depends on the particular system under study and can be readily appreciated by one of ordinary skill in the art.

When introducing elements of the present disclosure or the preferred embodiments(s) thereof, the articles “a,” “an,” “the,” and “said” are intended to mean that there are one or more of the elements. The terms “comprising,” “including,” and “having” are intended to be inclusive and mean that there may be additional elements other than the listed elements. [075]“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure

As used herein “ambient temperature” or “room temperature” is the temperature of the environment surrounding the process or experimental apparatus.

As used herein, the term “glass” refers to completely vitrified solids as well as to partially crystalline or glassy solids. Generally, a “glass” is a material below its glass transition temperature (T_{g}), as defined by for example differential scanning calorimetry (DSC) or dynamic mechanical analysis (DMA). Use temperatures defined as a range include all temperatures in which the swelling medium remains in the liquid phase. For aqueous media this may have a range including 0-100° C. For room temperature ionic liquids, as described herein, this may have a range from 0-160° C. Typically, the glassy domains may have a glass transition temperature of at least 60° C.

As used herein, the term “hydrogel” refers to a gel in which at least one liquid component is an aqueous medium or aqueous electrolyte.

As used herein, the term “gel polymer electrolyte” refers to a gel in which at least one liquid component is a non-aqueous medium or non-aqueous electrolyte.

It is understood that In some cases, a “hydrogel” can also be a “gel polymer electrolyte”.

As used herein, the term “monomer” refers to any chemical compound capable of forming a covalent bond with itself or a chemically different compound in a repetitive manner. The repetitive bond formation between monomers may lead to a linear, branched, super-branched, or three-dimensional product. Furthermore, monomers may themselves comprise repetitive building blocks, and when polymerized, the polymers formed from such monomers are then termed “block polymers.” Monomers may belong to various chemical classes of molecules including organic, organometallic, or inorganic molecules. The molecular weight of monomers may vary greatly range between about 4 Daltons and 20000 kDaltons. However, especially when monomers comprise repetitive building blocks, monomers may have even higher molecular weights. Monomers may also include additional reactive groups.

### II. Thermoplastic Elastomer Composites

The present disclosure provides thermoplastic elastomer composites. These thermoplastic elastomer composites comprise at least two styrenic or hydrogenated styrenic thermoplastic elastomers wherein the non-styrenic blocks of the at least two styrenic or hydrogenated styrenic thermoplastic elastomers comprises at least one block copolymer comprising at least one hydrophilic block and one of the at least two styrenic thermoplastic elastomers comprises at least one block copolymer comprising at least one hydrophobic block.

Block copolymers consist of two or more strands (“blocks” or “polymer blocks”) of different polymers chemically attached to each other. Properties of block copolymers herein can depend on copolymer sequence distribution, chemical nature of the blocks, average molecular weight, molecular weight distribution of the blocks and the copolymer, and any combination thereof.

A block copolymer is a polymer that consists of multiple different types of polymer chains that are covalently attached to each other. The monomers comprising a block copolymer are referred to as “A block”, “B block”, and a like. Accordingly, a diblock copolymer comprised of A and B blocks can be referred to as an AB block copolymer whereas triblock copolymer comprised of A, B, and C blocks can be referred to as an ABC block copolymer, a tetrablock copolymer comprised of A, B, C, and D blocks can be referred to as an ABCD block copolymer, and so on. AB block copolymer morphology is governed by three variables, volume fraction of one block (f_{A}) and the Flory-Huggins interaction parameter between the blocks (X_{AB}), and the overall polymer degree of polymerization or molecular weight. Adding just one block adds more variables which govern phase separation; ABC block copolymers are governed by the volume fractions of two blocks (which necessarily determine the third) (f_{A}, f_{B}) and the interaction parameters between all three blocks (X_{AB}, X_{AC}, X_{BC}), and the overall polymer degree of polymerization or molecular weight. In contrast to AB block copolymers, the added block in ABC block polymers allows for formation of additional morphologies and microstructures.

### (a) Styrenic Thermoplastic Elastomers

Styrenic thermoplastic elastomer composites described herein comprise at least two styrenic thermoplastic elastomers or the hydrogenated forms of those styrenic thermoplastic elastomers, By way of a non-limiting example, a hydrogenated styrenic block copolymer could contain the hydrogenated form polycyclohexylethylene. The non-styrenic blocks of the at least two styrenic or hydrogenated styrenic thermoplastic elastomers can be either hydrophobic or hydrophilic. The at least two styrenic thermoplastic elastomers each comprise diblock copolymers, triblock copolymers, tetrablock copolymers, or any combination thereof. By way of a non-limiting example, a styrenic thermoplastic elastomer of the present disclosure may comprise just a triblock copolymer or it may comprise both a diblock copolymer and a triblock copolymer.

### (b) Block Copolymer

Block copolymers described herein may be selected to impart certain properties and/or characteristics on the thermoplastic elastomer composites.

One of the at least two styrenic thermoplastic elastomers may comprise at least one block copolymer comprising at least one non-styrenic hydrophilic block. Generally, the hydrophilic block comprises at least one polyalkylene oxide block. Non-limiting examples of polyalkylenes for use in polyalkylene oxide block can include polyethylene oxide, polypropylene oxide, polybutylene oxide and the like.

The block copolymer used in compositions herein may comprise at least one polyalkylene block. In some embodiments, the polyalkylene oxide block herein may be polyethylene oxide (PEO) block. Generally, the PEO block may have an average molecular weight of 20 kDa to 800 kDa. In various embodiments, the PEO block may have an average molecular weight from about 20 kDa to about 25 kDa, about 25 kDa to about 30 kDa, about 30 kDa to about 35 kDa, from about 35 kDa to about 40 kDa from about 40 kDa to about 45 kDa, from about 45 kDa to about 50 kDa, from about 50 kDa to about 55 kDa, from about 55 kDa to about 60 kDa, from about 60 kDa to about 65 kDa, from about 65 kDa to about 70 kDa, from about 70 kDa to about 75 kDa, from about 75 kDa to about 80 kDa, from about 80 kDa to about 85 kDa, from about 85 kDa to about 90 kDa, from about 90 kDa to about 95 kDa, from about 95 kDa to about 100 kDa, from about 100 kDa to about 105 kDa, from about 105 kDa to about 110 kDa, from about 110 kDa to about 115 kDa, from about 115 kDa to about 120 kDa, from about 120 kDa to about 125 kDa, from about 125 kDa to about 130 kDa, from about 130 kDa to about 135 kDa, from about 135 kDa to about 140 kDa, from about 140 kDa to about 145 kDa, from about 145 kDa to about 150 kDa, from about 150 kDa to about 155 kDa, from about 155 kDa to about 160 kDa, from about 160 kDa to about 170 kDa, from about 170 kDa to about 180 kDa, from about 180 kDa to about 190 kDa, from about 190 kDa to about 200 kDa, from about 200 kDa to about 250 kDa, from about 250 kDa to about 300 kDa, from about 300 kDa to about 350 kDa, from about 350 kDa to about 400 kDa, from about 400 kDa to about 450 kDa, from about 450 kDa to about 500 kDa, from about 500 kDa to about 550 kDa, from about 550 kDa to about 600 kDa, from about 600 kDa to about 650 kDa, from about 650 kDa to about 700 kDa, or from about 750 kDa to about 800 kDa. In one embodiment, the PEO block may have an average molecular weight of greater than about 100 kDa.

One of the at least two styrenic thermoplastic elastomers may comprise at least one non-styrenic hydrophobic block. In general, the hydrophobic block comprises at least one hydrophobic non-glassy block. Non-limiting examples of hydrophobic non-glassy blocks comprise a polydiene, a hydrogenated polydiene, a polysiloxane, or any combinations thereof. In some embodiments the hydrophobic non-glassy blocks may comprise a substituted block, for example a substituted polydiene or a substituted polysiloxane.

The block copolymer used in compositions herein may comprise at least one polydiene block, or a hydrogenated form of a polydiene block. Polydienes are amorphous polymers having a glass transition temperature below room temperature, usually ranging between 170 and 250 K (−100° C. and −25° C.). Suitable non-limiting examples of polydienes can include polybutadiene (PB), polychloroprene, polyisoprene (PI), or hydrogenated forms of polydienes such as poly(ethylene butylene) (PEP), poly(ethyl ethylene (PEE) and the like.

The at least one hydrophobic non-glassy block used in composites herein may comprise at least one polydiene block. In general, polydiene block may have an average molecular weight of 20 kDa to 800 kDa. In general, the polydiene block may have an average molecular weight from about 20 kDa to about 25 kDa, about 25 kDa to about 30 kDa, about 30 kDa to about 35 kDa, from about 35 kDa to about 40 kDa from about 40 kDa to about 45 kDa, from about 45 kDa to about 50 kDa, from about 50 kDa to about 55 kDa, from about 55 kDa to about 60 kDa, from about 60 kDa to about 65 kDa, from about 65 kDa to about 70 kDa, from about 70 kDa to about 75 kDa, from about 75 kDa to about 80 kDa, from about 80 kDa to about 85 kDa, from about 85 kDa to about 90 kDa, from about 90 kDa to about 95 kDa, from about 95 kDa to about 100 kDa, from about 100 kDa to about 105 kDa, from about 105 kDa to about 110 kDa, from about 110 kDa to about 115 kDa, from about 115 kDa to about 120 kDa, from about 120 kDa to about 125 kDa, from about 125 kDa to about 130 kDa, from about 130 kDa to about 135 kDa, from about 135 kDa to about 140 kDa, from about 140 kDa to about 145 kDa, from about 145 kDa to about 150 kDa, from about 150 kDa to about 155 kDa, from about 155 kDa to about 160 kDa, from about 160 kDa to about 170 kDa, from about 170 kDa to about 180 kDa, from about 180 kDa to about 190 kDa, from about 190 kDa to about 200 kDa, from about 200 kDa to about 250 kDa, from about 250 kDa to about 300 kDa, from about 300 kDa to about 350 kDa, from about 350 kDa to about 400 kDa, from about 400 kDa to about 450 kDa, from about 450 kDa to about 500 kDa, from about 500 kDa to about 550 kDa, from about 550 kDa to about 600 kDa, from about 600 kDa to about 650 kDa, from about 650 kDa to about 700 kDa, or from about 750 kDa to about 800 kDa. In one embodiment, the polydiene block may have an average molecular weight of greater than about 100 kDa.

The block copolymer used in compositions herein may comprise at least one polysiloxane block. Generally, the polysiloxane block may have an average molecular weight of 20 kDa to 800 kDa. In various embodiments, the polysiloxane block may have an average molecular weight from about 20 kDa to about 25 kDa, about 25 kDa to about 30 kDa, about 30 kDa to about 35 kDa, from about 35 kDa to about 40 kDa from about 40 kDa to about 45 kDa, from about 45 kDa to about 50 kDa, from about 50 kDa to about 55 kDa, from about 55 kDa to about 60 kDa, from about 60 kDa to about 65 kDa, from about 65 kDa to about 70 kDa, from about 70 kDa to about 75 kDa, from about 75 kDa to about 80 kDa, from about 80 kDa to about 85 kDa, from about 85 kDa to about 90 kDa, from about 90 kDa to about 95 kDa, from about 95 kDa to about 100 kDa, from about 100 kDa to about 105 kDa, from about 105 kDa to about 110 kDa, from about 110 kDa to about 115 kDa, from about 115 kDa to about 120 kDa, from about 120 kDa to about 125 kDa, from about 125 kDa to about 130 kDa, from about 130 kDa to about 135 kDa, from about 135 kDa to about 140 kDa, from about 140 kDa to about 145 kDa, from about 145 kDa to about 150 kDa, from about 150 kDa to about 155 kDa, from about 155 kDa to about 160 kDa, from about 160 kDa to about 170 kDa, from about 170 kDa to about 180 kDa, from about 180 kDa to about 190 kDa, from about 190 kDa to about 200 kDa, from about 200 kDa to about 250 kDa, from about 250 kDa to about 300 kDa, from about 300 kDa to about 350 kDa, from about 350 kDa to about 400 kDa, from about 400 kDa to about 450 kDa, from about 450 kDa to about 500 kDa, from about 500 kDa to about 550 kDa, from about 550 kDa to about 600 kDa, from about 600 kDa to about 650 kDa, from about 650 kDa to about 700 kDa, or from about 750 kDa to about 800 kDa. In one embodiment, the polysiloxane block may have an average molecular weight of greater than about 100 kDa

The block copolymer used in compositions herein may comprise at least one polystyrene block (PS). The PS may be used in hydrophobic and/or hydrophilic blocks. In general, the PS may have an average molecular weight of 3 kDa to 800 kDa. In various embodiments, PS may have an average molecular weight of about 3 kDa to about 5 kDa, about 5 kDa to about 10 kDa, about 10 kDa to about 15 kDa, about 15 kDa to about 20 kDa, about 20 kDa to about 25 kDa, about 25 kDa to about 30 kDa, about 30 kDa to about 35 kDa, from about 35 kDa to about 40 kDa from about 40 kDa to about 45 kDa, from about 45 kDa to about 50 kDa, from about 50 kDa to about 55 kDa, from about 55 kDa to about 60 kDa, from about 60 kDa to about 65 kDa, from about 65 kDa to about 70 kDa, from about 70 kDa to about 75 kDa, from about 75 kDa to about 80 kDa, from about 80 kDa to about 85 kDa, from about 85 kDa to about 90 kDa, from about 90 kDa to about 95 kDa, from about 95 kDa to about 100 kDa, from about 100 kDa to about 105 kDa, from about 105 kDa to about 110 kDa, from about 110 kDa to about 115 kDa, from about 115 kDa to about 120 kDa, from about 120 kDa to about 125 kDa, from about 125 kDa to about 130 kDa, from about 130 kDa to about 135 kDa, from about 135 kDa to about 140 kDa, from about 140 kDa to about 145 kDa, from about 145 kDa to about 150 kDa, from about 150 kDa to about 155 kDa, from about 155 kDa to about 160 kDa, from about 160 kDa to about 170 kDa, from about 170 kDa to about 180 kDa, from about 180 kDa to about 190 kDa, from about 190 kDa to about 200 kDa, from about 200 kDa to about 250 kDa, from about 250 kDa to about 300 kDa, from about 300 kDa to about 350 kDa, from about 350 kDa to about 400 kDa, from about 400 kDa to about 450 kDa, from about 450 kDa to about 500 kDa, from about 500 kDa to about 550 kDa, from about 550 kDa to about 600 kDa, from about 600 kDa to about 650 kDa, from about 650 kDa to about 700 kDa, or from about 750 kDa to about 800 kDa. In one embodiment, the PS may have an average molecular weight of greater than about 3 kDa. In another embodiments, the PS may have an average molecular weight of less than 800 kDa.

The PS block may be completely or partially hydrogenated. A hydrogenated PS may yield cyclohexyl, cyclohexenyl, and/or cyclohexadienyl moieties. For example, the PS domain of the block copolymer may be based on the hydrogenated forms of styrenic monomers, such as vinyl cyclohexylethylene. In various embodiments, the hydrogenation of a PS herein may occur under increased partial pressure of hydrogen, with a catalyst, or without a catalyst. Suitable non-limiting catalysts include palladium, platinum, rhodium, ruthenium, nickel, or other transition metals. A catalyst may further comprise a support matrix, such as calcium carbonate (CaCO₃), carbon, porous silica, and a like. Suitable non-limiting examples of hydrogenation catalysts on supports include, but are not limited to, palladium on carbon, palladium on calcium carbonate, and platinum on porous silica.

The polydiene block may be completely or partially hydrogenated. A hydrogenated polydiene may yield ethyl ethylene or ethyl butylene moieties. For example, the polydiene domain of the block copolymer may be based on the hydrogenated forms of diene monomers, such as ethyl ethylene or ethyl butylene. In various embodiments, hydrogenation of a polydiene block may transform polybutadiene into polyethylethylene (PEE) or poly(ethylene butylene) (PEB), or polyisoprene into poly(ethylene alt propylene) (PEP).

In various embodiments, the hydrogenation of a polydiene herein may occur under increased partial pressure of hydrogen, with a catalyst, or without a catalyst. Suitable non-limiting catalysts include palladium, platinum, rhodium, ruthenium, nickel, or other transition metals. A catalyst may further comprise a support matrix, such as calcium carbonate (CaCO₃), carbon, porous silica, and a like. Suitable non-limiting examples of hydrogenation catalysts on supports include, but are not limited to, palladium on carbon, palladium on calcium carbonate, and platinum on porous silica.

### (c) Multiblock Copolymers

The present disclosure provides styrenic thermoplastic elastomers composites comprising multiblock (e.g., diblock, triblock, tetrablock, and so on) copolymers.

Diblock copolymers herein may contain at least two polymer blocks according to Formula (I):

A-B (I)

wherein A and B are polymer blocks. In some embodiments, A and B are the same polymer block. In other embodiments, A and B are different polymer blocks. A detailed description of examples of diblock copolymers can be found in U.S. Pat. Nos. 10,428,185 and 10,532,130, the contents of which are hereby incorporated by reference in their entirety.

The present disclosure provides styrenic thermoplastic elastomers composites comprising triblock copolymers. Triblock copolymers described herein may contain at least three polymer blocks according to Formula (II):

A-B-C (II);

wherein A, B, and C are polymer blocks. In various embodiments, the at least one of A, B, or C is a different polymer block than the other two. In other yet other some embodiments, the order of the polymer blocks is random. In still other embodiments, the order of the polymer blocks is specific.

The present disclosure provides styrenic thermoplastic elastomers composites comprising tetrablock copolymers. tetrablock copolymers described herein may contain at least four polymer blocks according to Formula (III):

A-B-C-D (III);

wherein A, B, C, and D are polymer blocks. In various embodiments, the at least one of A, B, C, or D is a different polymer block than the other three. In other yet other some embodiments, the order of the polymer blocks is random. In still other embodiments, the order of the polymer blocks is specific.

The present disclosure provides styrenic thermoplastic elastomers composites comprising pentablock copolymers. Pentablock copolymers described herein may contain at least five polymer blocks according to Formula (IV):

A-B-C-D-E (IV);

wherein A, B, C, and D and E are polymer blocks. In various embodiments, the at least one of A, B, C, D or E is a different polymer block than the other four. In yet other embodiments, the order of the polymer blocks is random. In still other embodiments, the order of the polymer blocks is specific.

The block copolymers may comprise polymer blocks ranging in number average molecular weight (Mn) and/or volume fraction of the final block copolymer. Methods of measuring Mn and/or volume fraction are known in the art and are suitable for use herein. Mn may be determined by proton nuclear magnetic resonance (¹H-NMR). Volume fractions (f) may be calculated from monomer weight and polymer densities at a desired temperature. In general, the desired temperate suitable for measuring volume fractions may be about 120° C. to about 150° C.

The block copolymers herein may comprise a polystyrene block wherein the volume fraction ranges from about 0.005 f to about 0.6 f. In various embodiments, block copolymers herein may comprise a polystyrene block (PS) wherein the volume fraction may be about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6 f. In one embodiment, the block copolymers herein may comprise a polystyrene block wherein the volume fraction may be about 0.005 to about 0.5 f.

Generally, the block copolymers herein may comprise a polydiene block or a hydrogenated polydiene block wherein the volume fraction ranges from about 0.1 f to about 0.9 f. In various embodiments, block copolymers herein may comprise a polydiene block wherein the volume fraction may be about 0.1, about 0.2, about 0.3, about 0.4, about 0.5, about 0.6, about 0.7, about 0.8, or about 0.9 f. In one embodiment, the block copolymers herein may comprise a polydiene block wherein the volume fraction may be about 0.1 to about 0.9 f.

Generally, the block copolymers herein may comprise a PEO block wherein the volume fraction ranges from about 0.1 f to about 0.9 f. In various embodiments, the block copolymers herein may comprise a PEO block wherein the volume fraction may be about 0.1, about 0.2, about 0.3, about 0.4, about 0.5, about 0.6, about 0.65, about 0.7, about 0.75, about 0.8, about 0.85, about or about 0.9 f. In one embodiment, the block copolymers herein may comprise a PEO block wherein the volume fraction may be about 0.1 to about 0.9 f.

Generally, the triblock copolymers herein may comprise a polydiene block or a hydrogenated polydiene block wherein the volume fraction ranges from about 0.1 f to about 0.9 f.

In general, the block copolymers herein may comprise a polybutadiene block wherein the volume fraction ranges from about 0.1 f to about 0.9 f. In various embodiments, the triblock copolymers herein may comprise a polybutadiene block wherein the volume fraction may be about 0.1, about 0.2, about 0.3, about 0.4, about 0.5, about 0.6, about 0.7, about 0.8, or about 0.9 f.

In an embodiment, one of the at least two styrenic thermoplastic elastomers comprising at least one block copolymer comprising at least one hydrophilic block is a polystyrene-polyethylene oxide-polystyrene triblock copolymer (SOS or PS-PEO-PS).

In another embodiment, one of the at least two styrenic thermoplastic elastomers comprising at least one block copolymer comprising at least one hydrophobic block is a polystyrene-butadiene-polystyrene triblock copolymer (SBS or PS-PB-PS).

In an embodiment, one of the at least two styrenic thermoplastic elastomers comprising at least one block copolymer comprising at least one hydrophobic block is a polystyrene-polyisoprene-polystyrene triblock copolymer (SIS or PS-PI-PS).

In an embodiment, one of the at least two styrenic thermoplastic elastomers comprising at least one block copolymer comprising at least one hydrophobic block is a polystyrene-poly(ethylene butylene)-polystyrene triblock copolymer (SEBS or PS-PEB-PS).

In an embodiment, one of the at least two styrenic thermoplastic elastomers comprising at least one block copolymer comprising at least one hydrophobic block is a polystyrene-polydimethylsiloxane-polystyrene triblock copolymer (SDS or PS-PDMS-PS).

In yet another embodiment, the at least two styrenic thermoplastic elastomers comprise SOS (PS-PEO-PS) and SBS (PS-PB-PS).

In yet another embodiment, the at least two styrenic thermoplastic elastomers comprise SOS (PS-PEO-PS) and SIS (PS-PI-PS).

In yet another embodiment, the at least two styrenic thermoplastic elastomers comprise SOS (PS-PEO-PS) and SEBS (PS-PEB-PS).

In yet another embodiment, the at least two styrenic thermoplastic elastomers comprise SOS (PS-PEO-PS) and SDS (PS-PDMS-PS).

In yet another embodiment, the at least two styrenic thermoplastic elastomers comprise SBS (PS-PB-PS) and SDS (PS-PDMS-PS).

In yet another embodiment, the at least two styrenic thermoplastic elastomers comprise SEBS (PS-PEB-PS) and SDS (PS-PDMS-PS).

In yet another embodiment, the at least two styrenic thermoplastic elastomers comprise SIS (PS-PI-PS) and SDS (PS-PDMS-PS).

In yet another embodiment, the at least two styrenic thermoplastic elastomers comprise SOS (PS-PEO-PS), SBS (PS-PB-PS) and SDS (PS-PDMS-PS).

In yet another embodiment, the at least two styrenic thermoplastic elastomers comprise SOS (PS-PEO-PS), SEBS (PS-PEB-PS) and SDS (PS-PDMS-PS).

In yet another embodiment, the at least two styrenic thermoplastic elastomers comprise SOS (PS-PEO-PS), SIS (PS-PI-PS) and SDS (PS-PDMS-PS).

The block copolymer species (e.g., SOS, SBS, SIS, SEBS, SDS) undergo a self-assembly process when heated or thermally processed, in which they organize into a periodic nanostructure of spherical or cylindrical domains on the order of 10-20 nm in size (very small). Without being bound to any one theory, it is believed that the unique mechanical performance and durability of the composite is the result of the shared styrenic (PS) blocks in the two systems helping to compatibilize the interface between the two components in the composites.

In general, the ratio of the at least two styrenic thermoplastic elastomers comprising at least one block copolymer comprising at least one non-styrenic hydrophilic block to the one of the at least two styrenic thermoplastic elastomers comprising at least one block copolymer comprising at least one non-styrenic hydrophobic block may be 1:99 to 99:1. In various embodiments, the ratio of the one of the at least two styrenic thermoplastic elastomers comprising at least one block copolymer comprising at least one non-styrenic hydrophilic block to the one of the at least two styrenic thermoplastic elastomers comprising at least one block copolymer comprising at least one non-styrenic hydrophobic block is 1:19, 1:1, or 19:1. In yet other embodiments, the ratio of the one of the at least two styrenic thermoplastic elastomers comprising at least one block copolymer comprising at least one non-styrenic hydrophilic block to the one of the at least two styrenic thermoplastic elastomers comprising at least one block copolymer comprising at least one non-styrenic hydrophobic block is 1:4, 1:3, 1:2, 1:1, 1:2, 1:3, or 4:1.

Generally, the ratio of the triblock copolymers, SOS to SBS, is 1:19 to 19:1. In various embodiments, the ratio of the triblock copolymers, SOS to SBS, is 1:4, 1:3, 1:2, 1:1, 1:2, 1:3, or 4:1.

Generally, the ratio of the triblock copolymers, SOS to SEBS, is 1:19 to 19:1. In various embodiments, the ratio of the triblock copolymers, SOS to SEBS, is 1:4, 1:3, 1:2, 1:1, 1:2, 1:3, or 4:1.

Generally, the ratio of the triblock copolymers, SOS to SDS, is 1:19 to 19:1. In various embodiments, the ratio of the triblock copolymers, SOS to SDS, is 1:4, 1:3, 1:2, 1:1, 1:2, 1:3, or 4:1.

### III. Methods of Preparing the Thermoplastic Elastomer (TPE) Composites

The present disclosure provides a method for preparing a thermoplastic elastomer (TPE) composite. The method comprises contacting the at least two thermoplastic elastomers in a molar ratio from between 1:99 and 99:1 to form a TPE dry blend. The TPE dry blend is heated under conditions mechanical mixing, mechanical extrusion or mechanical pressure to form a TPE composite melt. The TPE composite melt is allowed to attain ambient temperature to form an TPE composite.

### (a) Blending of the Thermoplastic Elastomer (TPE) Dry Blend

The TPE dry blend may be formed by dissolving the at least two thermoplastic elastomers in at least one organic solvent and removing the at least one organic solvent. The organic solvent may be a polar protic solvent, a polar aprotic solvent, a non-polar solvent, or combinations thereof. Suitable examples of polar protic solvents include, but are not limited to alcohols such as methanol, ethanol, isopropanol, n-propanol, isobutanol, n-butanol, s-butanol, t-butanol, and the like; diols such as propylene glycol; organic acids such as formic acid, acetic acid, and so forth; amines such as trimethylamine, or triethylamine, and the like; amides such as formamide, acetamide, and so forth; and combinations of any of the above. Non-limiting examples of suitable polar aprotic solvents include acetonitrile, dichloromethane (DCM), diethoxymethane, N,N-dimethylacetamide (DMAC), N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, 1,4-dioxane, N-methyl-2-pyrrolidinone (NMP), ethyl formate, formamide, hexamethylphosphoramide, N-methylacetamide, N-methylformamide, methylene chloride, nitrobenzene, nitromethane, propionitrile, sulfolane, tetramethylurea, tetrahydrofuran (THF), 2-methyltetrahydrofuran, trichloromethane, and combinations thereof. Suitable examples of non-polar solvents include, but are not limited to, alkane and substituted alkane solvents (including cycloalkanes), aromatic hydrocarbons, esters, ethers, combinations thereof, and the like. Specific non-polar solvents that may be employed include, for example, benzene, butyl acetate, t-butyl methylether, chlorobenzene, chloroform, chloromethane, cyclohexane, dichloromethane, dichloroethane, diethyl ether, ethyl acetate, diethylene glycol, fluorobenzene, heptane, hexane, isopropyl acetate, methyltetrahydrofuran, pentyl acetate, n-propyl acetate, tetrahydrofuran, toluene, and combinations thereof. In particular, the solvent may be benzene or toluene.

The TPE dry blend may be formed by dissolution in at least one solvent at concentration that may be between about 1 wt % TPE dry blend and about 20 wt % TPE dry blend, such as between 1 wt % TPE dry blend and 2 wt % TPE dry blend, such as between 2 wt % TPE dry blend and 3 wt % TPE dry blend, such as between 3 wt % TPE dry blend and 4 wt % TPE dry blend, such as between 4 wt % TPE dry blend and 5 wt % TPE dry blend, such as between 5 wt % TPE dry blend and 6 wt % TPE dry blend, such as between 6 wt % TPE dry blend and 7 wt % TPE dry blend, such as between 7 wt % TPE dry blend and 8 wt % TPE dry blend, such as between 8 wt % TPE dry blend and 9 wt % TPE dry blend, such as between 9 wt % TPE dry blend and 10 wt % TPE dry blend, such as between 10 wt % TPE dry blend and 11 wt % TPE dry blend, such as between 11 wt % TPE dry blend and 12 wt % TPE dry blend, such as between 12 wt % TPE dry blend and 13 wt % TPE dry blend, such as between 13 wt % TPE dry blend and 14 wt % TPE dry blend, such as between 14 wt % TPE dry blend and 15 wt % TPE dry blend, such as between 15 wt % TPE dry blend and 16 wt % TPE dry blend, such as between 16 wt % TPE dry blend and 17 wt % TPE dry blend, such as between 17 wt % TPE dry blend and 18 wt % TPE dry blend, such as between 18 wt % TPE dry blend and 19 wt % TPE dry blend, or such as between 19 wt % TPE dry blend and 20 wt % TPE dry blend. The concentration may be between about 5 wt % and 15 wt %, or about 10 wt %.

In the TPE dry blend, the molar ratio of the at least two thermoplastic elastomers may be between about 99:1 and about 1:99, such as between about 99:1 and about 95:5, such as between about 95:5 and about 90:10, such as between about 90:10 and about 85:15, such as between about 85:15 and about 80:20, such as between about 80:20 and about 75:25, such as between about 75:25 and about 70:30, such as between about 70:30 and about 65:35, between about 65:35 and about 60:40, between about 60:40 and about 55:45, between about 55:45 and about 50:50, between about 50:50 and about 55:45, between about 55:45 and about 45:65, between about 45:65 and about 40:60, between about 40:60 and about 35:65, between about 35:65 and about 30:70, between about 30:70 and about 25:75 between about 25:75 and about 20:80=, between about 20:80 and about 15:85, between about 15:85 and about 10:90, between about 10:90 and about 5:95, or between about 5:95 and about 1:99. In particular, the molar ratio may be between about 80:20 and about 20:80, between about 70:30 and about 30:70, between about 60:40 and about 40:60 or at about 50:50. The molar ratio may also be about 4:96, about 3:97, about 2:98, or about 1:99.

### (b) Heating of the Thermoplastic Elastomer Dry Blend

In one embodiment, the thermoplastic elastomer dry blend is processed under a combination of pressure and heat for a period of time to form a TPE composite. The TPE dry blend may be heated to a temperature between about 80° C. and about 250° C., such as between about 80° C. and about 90° C., such as between about 90° C. and about 100° C., such as between about 100° C. and about 110° C., between about 110° C. and about 120° C., between about 120° C. and about 130° C., between about 130° C. and about 140° C., between about 140° C. and about 150° C., between about 150° C. and about 160° C., between about 160° C. and about 170° C., between about 160° C. and about 170° C., between about 170° C. and about 180° C., between about 180° C. and about 190° C., between about 190° C. and about 200° C., between about 200° C. and about 210° C., between about 210° C. and about 220° C., between about 220° C. and about 230° C., between about 230° C. and about 240° C., between about 240° C. and about 250° C., between about 250° C. and about 260° C., between about 260° C. and about 270° C., between about 260° C. and about 270° C., between about 270° C. and about 280° C., between about 280° C. and about 290° C., between about 290° C. and about 300° C., between about 300° C. and about 310° C., or between about 310° C. and about 320° C. The temperature may be between about 140° C. and about 160° C., such as about 150° C.

The TPE dry blend may be heated without or without pressure. If heated under pressure, the TPE dry blend may be heated under a pressure between about 1000 lbf and about 25000 lbf, such as between about 1000 lbf and about 2000 lbf, between about 2000 lbf and about 3000 lbf, between about 3000 lbf and about 4000 lbf, between about 4000 lbf and about 50000 lbf, between about 5000 lbf and about 6000 lbf, between about 6000 lbf and about 7000 lbf, between about 7000 lbf and about 8000 lbf, between about 8000 lbf and about 9000 lbf, between about 9000 lbf and about 10000 lbf, between about 10000 lbf and about 11000 lbf, between about 11000 lbf and about 12000 lbf, between about 12000 lbf and about 13000 lbf, between about 13000 lbf and about 14000 lbf, between about 14000 lbf and about 15000 lbf, between about 15000 lbf and about 16000 lbf, between about 16000 lbf and about 17000 lbf, between about 17000 lbf and about 18000 lbf, between about 18000 lbf and about 19000 lbf, between about 19000 lbf and about 20000 lbf, between about 20000 lbf and about 21000 lbf, between about 21000 lbf and about 22000 lbf, between about 22000 lbf and about 23000 lbf, between about 23000 lbf and about 24000 lbf, between about 24000 lbf and about 25000 lbf, or at about 15000 lbf. The pressure may be between about 10000 lbf and about 20000 lbf, such as about 15000 lbf.

Additionally, pressure may be applied to samples of the TPE dry blend placed in a vacuum bag, such that a dynamic reduced pressure of at least 15 Torr inside the bag is achieved during heating with or without pressure. That is, the sample may be placed into a vacuum bag during operation of the press used to heat and squeeze the sample. Doing so has been discovered herein to reduce the number of microbubbles, as well as grain boundary and particle sintering defects in the melt.

Additionally, mechanical mixing may be applied to samples of the TPE dry blend using an extruding or microcompounding device. That is, the sample may be placed into a twin screw extruder during heating. Doing so has been discovered control the domain sizes achievable in the TPE composite of at least two thermoplastic elastomers. If placed in an extruding device such as a twin screw extruder, the TPE dry blend may be processed using screw speeds between about 50 rpm and about 250 rpm, such as between about 50 rpm and about 60 rpm, such as between about 60 rpm and about 70 rpm, such as between about 70 rpm and about 80 rpm, such as between about 80 rpm and about 90 rpm, such as between about 90 rpm and about 100 rpm, such as between about 100 rpm and about 110 rpm, between about 110 rpm and about 120 rpm, between about 120 rpm and about 130 rpm, between about 130 rpm and about 140 rpm, between about 140 rpm and about 150 rpm, between about 150 rpm and about 160 rpm, between about 160 rpm and about 170 rpm, between about 160 rpm and about 170 rpm, between about 170 rpm and about 180 rpm, between about 180 rpm and about 190 rpm, between about 190 rpm and about 200 rpm, between about 200 rpm and about 210 rpm, between about 210 rpm and about 220 rpm, between about 220 rpm and about 230 rpm, between about 230 rpm and about 240 rpm, or between about 240 rpm and about 250 rpm. The temperature may be between about 100 rpm and about 200 rpm, such as about 150 rpm.

The TPE dry blend may be heated with or without pressure, or with or without mechanical mixing for between about 5 minutes and about 60 minutes, such as between about 5 minutes and about 10 minutes, between about 10 minutes and about 15 minutes, between about 15 minutes and about 20 minutes, between about 20 minutes and about 25 minutes, between about 25 minutes and about 30 minutes, between about 30 minutes and about 35 minutes, between about 35 minutes and about 40 minutes, between about 40 minutes and about 45 minutes, between about 45 minutes and about 50 minutes, between about 50 minutes and about 50 minutes or between about 55 minutes and about 60 minutes. In particular, the SO—SOS dry blend may be heated for about 15 minutes, or for about 5 minutes.

The heating may occur in heating-cooling cycles, wherein the TPE dry blend is heated for a period of time and then allowed to cool to ambient temperature before re-heating. For example, the TPE dry blend may be heated for a period of 5 minutes and then allowed to cool to ambient temperature before reheating. Generally, the dry blend may pass through 1 to 10 cycles. Any combination of these features may be used for processing the TPE dry blend. For example, the TPE dry blend may be heated at 150° C. at 5000 lbf in a vacuum bag for 4 heating-cooling cycles.

The methods used to produce the TPE composite solid influence the TPE composite microstructure, particularly the average domain sizes of the constituent TPEs. Without being bound by any one theory, it is understood that the average size of the TPE domains can influence the mechanical, physical, or chemical properties exhibited by the TPE composite, or the TPE hydrogel composites or TPE gel polymer electrolyte composites subsequently formed. That is, the use of solvent to form the TPE dry blend, the application of heat to the TPE dry blend, the application of pressure to the TPE dry blend, and/or the application of mechanical mixing to the TPE dry blend can be used to produce a particular microstructure.

The TPE dry blend may have a microstructure characterized by TPE domains of sizes of about 0.1 microns to about 50 microns. In some embodiments the TPE dry blend may have a microstructure characterized by TPE domains of sizes of about 0.1 microns to about 5 microns, about 5 microns to about 10 microns, about 10 microns to about 15 microns, about 15 microns to about 20 microns, about 20 microns to about 25 microns, about 25 microns to about 30 microns, about 30 microns to about 35 microns, about 35 microns to about 40 microns, about 40 microns to about 45 microns, or about 45 microns to about 50 microns. In some embodiments the TPE dry blend may have a microstructure characterized by TPE domains of sizes of about 0.1 microns to about 20 microns. In some embodiments the TPE dry blend may have a microstructure characterized by TPE domains of sizes of about 0.1 microns to about 10 microns.

### IV. Thermoplastic Elastomer (TPE) Hydrogel Composites

Another aspect of the present disclosure provides for thermoplastic elastomer hydrogel composites. The thermoplastic elastomer hydrogel composites comprise (a) any one of the thermoplastic elastomer composites as described in Sections II and III; and (b) at least one aqueous liquid medium comprising one or more aqueous solvents, aqueous liquid electrolytes, or a combination thereof.

The thermoplastic elastomer composites are described in more detail in Section (II and III) above.

The liquid medium is utilized with the thermoplastic elastomer composites to prepare the thermoplastic elastomer hydrogel composites. The liquid medium comprises one or more aqueous solvents, aqueous liquid electrolytes, or a combination thereof.

In general, the liquid medium may comprise one or more solvents in combination with water. The one or more solvents may be a polar protic solvent, a polar aprotic solvent, a non-polar solvent, or combinations thereof. Suitable examples of polar protic solvents include but are not limited to water; alcohols such as methanol, ethanol, isopropanol, n-propanol, iso-butanol, n-butanol, s-butanol, t-butanol, and the like; diols such as propylene glycol; organic acids such as formic acid, acetic acid, and so forth; amines such as trimethylamine, or triethylamine, and the like; amides such as formamide, acetamide, and so forth; and combinations of any of the above. Non-limiting examples of suitable polar aprotic solvents include acetonitrile, dichloromethane (DCM), diethoxymethane, N,N-dimethylacetamide (DMAC), N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, 1,4-dioxane, N-methyl-2-pyrrolidinone (NMP), ethyl formate, formamide, hexamethylphosphoramide, N-methylacetamide, N-methylformamide, methylene chloride, nitrobenzene, nitromethane, propionitrile, sulfolane, tetramethylurea, tetrahydrofuran (THF), 2-methyltetrahydrofuran, trichloromethane, and combinations thereof. Suitable examples of non-polar solvents include, but are not limited to, alkane and substituted alkane solvents (including cycloalkanes), aromatic hydrocarbons, esters, ethers, combinations thereof, and the like. Specific non-polar solvents that may be employed include, for example, benzene, butyl acetate, t-butyl methyl ether, chlorobenzene, chloroform, chloromethane, cyclohexane, dichloromethane, dichloroethane, diethyl ether, ethyl acetate, diethylene glycol, fluorobenzene, heptane, hexane, isopropyl acetate, methyl tetrahydrofuran, pentyl acetate, n-propyl acetate, tetrahydrofuran, toluene, and combinations thereof.

In one embodiment, the solvent may be water. The water may further comprise a buffer, such as phosphate-buffered saline (PBS) or Ringer's solution, or a like.

In general, the TPE hydrogel composite may have a liquid content (for example water content) ranging from 0 w/v % to about 95 w/v %. The TPE hydrogel composite may have a liquid content ranging from 0.01 w/v % to about 95 w/v %. In various embodiments, the TPE hydrogel may have a liquid content ranging from 0 w/v % to about 85 w/v %, from 0 w/v % to about 5 w/v %, from about 5 w/v % to about 10 w/v %, from about 10 w/v % to about 15 w/v %, from about 15 w/v % to about 20 w/v %, from about 20 w/v % to about 25 w/v %, from about 25 w/v % to about 30 w/v %, from about 30 w/v % to about 35 w/v %, from about 35 w/v % to about 40 w/v %, from about 40 w/v % to about 45 w/v %, from about 45 w/v % to about 50 w/v %, from about 50 w/v % to about 55 w/v %, from about 55 w/v % to about 60 w/v %, from about 65 w/v % to about 70 w/v %, from about 70 w/v % to about 75 w/v %, from about 75 w/v % to about 80 w/v %, from about 80 w/v % to about 85 w/v %, from about 85 w/v % to about 90 w/v %, or from about 90 w/v % to about 95 w/v %.

The TPE hydrogel composites have some unique and unexpected properties. The TPE hydrogel composites have a lubricious surface meaning the surface is smooth, glassy in appearance, and slippery with a low coefficient of friction.

Generally, the TPE hydrogels have a bulk modulus averaged over the initial 10% strain of about 0.1 megapascals (MPa) to about 25 MPa. In various embodiments, the TPE hydrogels have a bulk modulus averaged over the initial 10% strain of about 0.1 megapascals (MPa) to about 25 MPa. In various embodiments, the TPE hydrogel composites have a modulus of about 0.1 MPa to about 1 MPa, from about 1 MPa to about 2 MPa, from about 2 MPa to about 3 MPa, from about 3 MPa to about 4 MPa, from about 4 MPa to about 5 MPa, from about 5 MPa to about 6 MPa, from about 6 MPa to about 7 MPa, from about 7 MPa to about 8 MPa, from about 8 MPa to about 9 MPa, from about 9 MPa to about 10 MPa, from about 10 MPa to about 11 MPa, from about 11 MPa to about 12 MPa, from about 12 MPa to about 13 MPa, from about 13 MPa to about 14 MPa, from about 14 MPa to about 15 MPa, from about 15 MPa to about 16 MPa, from about 16 MPa to about 17 MPa, from about 17 MPa to about 18 MPa, from about 18 MPa to about 19 MPa, from about 19 MPa to about 20 MPa, from about 20 MPa to about 21 MPa, from about 21 MPa to about 22 MPa, from about 22 MPa to about 23 MPa, from about 23 MPa to about 24 MPa, or from about 24 MPa to about 25 MPa.

In general, the TPE hydrogel composites have a toughness of about 1 MJ/m³ to about 120 MJ/m³. In various embodiments, the TPE hydrogel composites have a toughness of about 1 MJ/m³ to about 5 MJ/m³, from about 5 MJ/m³ to about 10 MJ/m³, from about 10 MJ/m³ to about 20 MJ/m³, from about 20 MJ/m³ to about 30 MJ/m³, from about 30 MJ/m³ to about 40 MJ/m³, from about 40 MJ/m³ to about 50 MJ/m³, from about 50 MJ/m³ to about 60 MJ/m³, from about 60 MJ/m³ to about 70 MJ/m³, from about 70 MJ/m³ to about 80 MJ/m³, from about 80 MJ/m³ to about 90 MJ/m³, from about 90 MJ/m³ to about 100 MJ/m³, from about 100 MJ/m³ to about 110 MJ/m³, or from about 110 MJ/m³ to about 120 MJ/m³.

With the above properties, the TPE hydrogel composites are resistant to biofouling, fatigue, wear, fracture, degradation, or any combination thereof.

In one embodiment, the TPE hydrogel composite may have a fatigue resistance to at least 500,000 compression cycles, such as at least 600,000 compression cycles, such as at least 700,000 compression cycles, such as at least 800,000 compression cycles, such as at least 900,000 compression cycles, such as at least 1,000,000 compression cycles, such as at least 1,500,000 compression cycles, such as at least 2,000,000 compression cycles, such as at least 2,500,000 compression cycles, such as at least 3,000,000 compression cycles, such as at least 3,500,000 compression cycles, such as at least 4,000,000 compression cycles, such as at least 4,500,000 compression cycles, such as at least 5,000,000 compression cycles, or such as at least 10,000,000 compression cycles. In counting the number of compression cycles, the cycles are preferably continuous, but need not be so, having a resting period between shorter runs of cycles.

The compression cycles may operate with at least 30% compression at a frequency of about 2 Hz. The fatigue resistance is characterized by a modulus recoverable to at least 80% of its value before the compression cycles were run, such as to at least 90%, to at least 95% or to at least 99% of its value before the compression cycles were run.

### V. Methods of Preparing the TPE Hydrogel Composite

Another aspect of the present disclosure provides for methods of preparing the TPE hydrogel composites. The methods comprise contacting the at least two thermoplastic elastomers in a molar ratio from between 1:99 and 99:1 to form a TPE dry blend. The TPE dry blend is heated under conditions mechanical mixing, mechanical extrusion or mechanical pressure to form a TPE composite melt. The TPE composite melt is allowed to attain ambient temperature to form an TPE composite. The TPE composite is then contacted with a liquid medium to form a TPE hydrogel composite.

The liquid medium may be an aqueous solvent, and aqueous liquid electrolytes, or a combination thereof. For example, any liquid medium described herein may be used.

The TPE composite may be contacted with the liquid medium at a temperature above −10° C. and below about 160° C., such as above 0° C. and below about 50° C., or at about 25° C. The temperature may be between about −10° C. and about −5° C., between about −5° C. and about 0° C., between about 0° C. and about 5° C., between about 5° C. and about 10° C., between about 10° C. and about 15° C., between about 15° C. and about 20° C., between about 20° C. and about 25° C., between about 25° C. and about 30° C., between about 30° C. and about 35° C., between about 35° C. and about 40° C., between about 40° C. and about 45° C., between about 45° C. and about 50° C., between about 50° C. and about 55° C., between about 55° C. and about 60° C., between about 60° C. and about 65° C., between about 65° C. and about 70° C., between about 70° C. and about 75° C., between about 75° C. and about 80° C., between about 80° C. and about 85° C., between about 85° C. and about 90° C., between about 90° C. and about 95° C., between about 95° C. and about 100° C., between about 100° C. and about 105° C., between about 105° C. and about 110° C., between about 110° C. and about 115° C., between about 115° C. and about 120° C., between about 120° C. and about 125° C., between about 125° C. and about 130° C., between about 130° C. and about 135° C., between about 135° C. and about 140° C., between about 140° C. and about 145° C., between about 145° C. and about 150° C., between about 150° C. and about 155° C., or between about 155° C. and about 160° C.

### VI. Biomedical Applications

The TPE hydrogel composites may be used as hydrated adhesives, coating materials, elastic separation membranes for protein assemblies and biologics, and mechanical energy absorbers.

Another aspect of the present disclosure provides biomedical devices and implants prepared using the thermoplastic elastomer hydrogel composites detailed above. Such biomedical devices may include but are not limited to: wound healing dressings, medical tubing (such as catheters), medical bags, medical containers, medical leads (such as a cardiac electrical lead), medical device coatings, or medical implants. Biomedical implants may include, but are not limited to soft tissue replacements such as intervertebral discs, meniscus, labria, or fibrocartilage.

One example of a biomedical implant is a synthetic meniscus replacement comprising a crescent shaped disk replacement prepared using the thermoplastic elastomer hydrogel composites detailed above. The thermoplastic elastomer hydrogel composites may be shaped or printed using an injection-molder or a 3D printer into the specific size of the synthetic meniscus replacement for each individual patient, the physician performing the procedure, and/or the procedure.

The beneficial properties of the disclosed thermoplastic elastomer hydrogel composites such as natural biofouling resistance, intrinsic lubricity, low coefficients of friction and drug delivery capability, make them especially suited for biomedical applications.

Additionally, the ability to co-extrude the disclosed composites with defined concentrations of therapeutic agents, or to impregnate the hydrated domains simply by swelling in the presence of such agents provides a range of opportunities in the biomedical device market. Such opportunities include, for example, the integration of stent delivery with the simultaneous localized delivery of anti-clotting agents using a catheter manufactured in a single extrusion step is a technology.

### VII. Thermoplastic Elastomer Gel Polymer Electrolyte Composites

Another aspect of the present disclosure provides for thermoplastic elastomer gel polymer electrolyte composites. The thermoplastic elastomer gel polymer electrolyte composites comprise (a) any one of the thermoplastic elastomer composites as described in Section (II and III) wherein at least one of the one or more thermoplastic elastomers comprises at least one liquid electrolyte swellable component block; and (b) at least one non-aqueous liquid medium comprising one or more non-aqueous (organic) solvents, non-aqueous liquid electrolytes, or a combination thereof.

The thermoplastic elastomer composites are described in more detail in Section (II and III) above.

The liquid medium is utilized with the thermoplastic elastomer composites to prepare the thermoplastic elastomer gel polymer electrolyte composites. The liquid medium comprises one or more non-aqueous solvents, non-aqueous liquid electrolytes, or a combination thereof.

The TPE GPE composite described herein comprise one or more styrenic thermoplastic elastomers. At least one of the one or more styrenic thermoplastic elastomers comprises at least one liquid electrolyte swellable component block. The at least one liquid electrolyte swellable component block comprises diblock copolymers, triblock copolymers, tetrablock copolymers, or any combination thereof. By way of a non-limiting example, at least one liquid electrolyte swellable component block of the present disclosure may comprise just a triblock copolymer or it may comprise both a diblock copolymer and a triblock copolymer.

Block copolymers described herein may be selected to impart certain properties and/or characteristics on the TPE gel polymer electrolyte composite. The styrenic thermoplastic elastomer comprising at least one liquid electrolyte swellable component block of the disclosed TPE GPE composite, comprises at least one block copolymer comprising at least one hydrophilic block as described above in Section II. The GPE may also further comprise a styrenic thermoplastic elastomer comprising at least one hydrophobic block as described in Section II.

The liquid medium of the TPE GPE composites may comprise a liquid electrolyte. Suitable liquid electrolytes include imidazolium-based ionic liquids.

The liquid electrolyte medium may further comprise one or more solvents, liquid electrolytes, or a combination thereof.

In general, the liquid electrolyte medium may further comprise one or more solvents.

The non-aqueous electrolyte may be a room-temperature ionic liquid (RTIL), which are relatively non-volatile, highly tunable molten salts whose melting points are below ambient temperature. RTILs are solvents with low viscosities (10-100 cP), low melting points, a range of densities, and relatively small molar volumes. Generally, RTILs consist of a cation and an anion.

The cation in the RTIL may be imidazolium, phosphonium, ammonium, and pyridinium. In particular embodiments, the RTIL comprises an imidazolium cation; that is, the RTIL is an imidazolium-based ionic liquid. Each cation may be substituted with one or more R groups, such as an imidazolium having the formula [Rmim] or [R₂mim], wherein “mim” references the imidiazolium. The R group may comprise one or more n-alkyl, branched alkyl, alkenyl, such as vinyl or allyl, alkynyl, fluoroalkyl, benzyl, styryl, hydroxyl, ether, amine, nitrile, silyl, siloxy, oligo(ethylene glycol), isothiocyanates, and sulfonic acids. In particular, the R group may be an alkyl selected from methyl or ethyl.

The RTIL may be functionalized with one, two, three, or more oligo(alkylene glycol) substituents, such as an oligo(ethylene glycol). Alternatively, the oligo(alkylene glycol) may be a methylene glycol or a propylene glycol.

A vicinal diol substituent on the RTILs may provide greater aqueous solubility and possible water miscibility.

Polymerizable RTILs may be provided choosing one or more R groups on the cation from a styrene, vinyl, allyl, or other polymerizable group.

Examples of suitable cations in the RTIL include, but are not limited to, 1-ethyl-3-methyl imidazolium ([EMIM]), 1-hexyl-3-methyl imidazolium ([HMIM]), 1-vinyl-3-ethyl-imidazolium ([VEIM]), 1-allyl-3-methyl-imidazolium ([AMIM]), 1-hexyl-3-butyl-imidazolium ([HBIM]), 1-vinyl-3-methylimidazolium ([VMIM]), 1-hydroxyundecanyl-3-methylimidazolium ([(C₁₁OH)MIM]), tetrabutylphosphonium ([P4444]), 1-(2,3-dihydroxypropyl)-alkyl imidazolium ([(dhp)MIM]), and combinations thereof. For example, the cation may be 1-ethyl-3-methyl imidazolium ([EMIM]). The cation may be 1-hexyl-3-methyl imidazolium ([HMIM]). The cation may be 1-vinyl-3-ethyl-imidazolium ([VEIM]). The cation may be 1-allyl-3-methyl-imidazolium ([AMIM]). The cation may be 1-hexyl-3-butyl-imidazolium ([HBIM]), 1-vinyl-3-methylimidazolium ([VMIM]). The cation may be 1-hydroxyundecanyl-3-methylimidazolium ([(C₁₁OH)MIM]). The cation may be tetrabutylphosphonium ([P4444]). The cation may also be 1-(2,3-dihydroxypropyl)-alkyl imidazolium ([(dhp)MIM]).

Suitable anions (X) in the RTIL include, but are not limited to, triflate (OTf), dicyanamide (DCA), tricyanomethanide (TCM), tetrafluoroborate (BF4), hexafluorophosphate (PF6), taurinate (Tau), and bis(trifluoromethane)sulfonimide (TSFI). For example, the anion may be triflate (OTf). The anion may be dicyanamide (DCA). The anion may be tricyanomethanide (TCM). The anion may be tetrafluoroborate (BF4). The anion may be hexafluorophosphate (PF6). The anion may be taurinate (Tau). The anion may be bis(trifluoromethane)sulfonimide (TSFI).

Any combination of cations and anions described herein may be used to form a suitable RTIL. Examples of suitable RTILs include, but are not limited to, 1-ethyl-3-methyl imidazolium bis(trifluoromethane)sulfonamide ([EMIM][TFSI]), 1-hexyl-3-methyl imidazolium bis(trifluoromethane)sulfonamide ([HMIM][TFSI]), 1-vinyl-3-ethyl-imidazolium bis(trifluoromethane)sulfonamide ([VEIM][TFSI]), 1-allyl-3-methyl-imidazolium bis(trifluoromethane)sulfonamide ([AMIM][TFSI]), 1-hexyl-3-butyl-imidazolium bis(trifluoromethane)sulfonamide ([HBIM][TFSI]), 1-vinyl-3-methylimidazolium bis(trifluoromethane)sulfonamide ([VMIM][TFSI]), 1-hydroxyundecanyl-3-methylimidazolium bis(trifluoromethane)sulfonamide ([(C₁₁OH)MIM][TFSI]), 1-ethyl-3-methylimidazolium tricyanomethanide ([EMIM][TCM]), tetrabutylphosphonium taurinate, ([P4444][Tau]), 1-ethyl-3-methylimidazolium dicyanamide ([EMIM][DCA]), 1-(2,3-dihydroxypropyl)-alkyl imidazolium dicyanamide ([(dhp)MIM][DCA]), 1-(2,3-dihydroxypropyl)-3-alkyl imidazolium tetrafluoroborate ([(dhp)MIM][BF4]), 1-(2,3-dihydroxypropyl)-3-alkyl imidazolium bis(trifluoromethane)sulfonimide ([(dhp)MIM][TFSI]), 1-(2,3-dihydroxypropyl)-3-alkyl imidazolium hexafluorophosphate ([(dhp)MIM][PF6]), or combinations thereof.

For example, the RTIL may be 1-ethyl-3-methyl imidazolium bis(trifluoromethane)sulfonamide ([EMIM][TFSI]). The RTIL may be 1-hexyl-3-methyl imidazolium bis(trifluoromethane)sulfonamide ([HMIM][TFSI]). The RTIL may be 1-vinyl-3-ethyl-imidazolium bis(trifluoromethane)sulfonamide ([VEIM][TFSI]). The RTIL may be 1-allyl-3-methyl-imidazolium bis(trifluoromethane)sulfonamide ([AMIM][TFSI]). The RTIL may be 1-hexyl-3-butyl-imidazolium bis(trifluoromethane)sulfonamide ([HBIM][TFSI]). The RTIL may be 1-vinyl-3-methylimidazolium bis(trifluoromethane)sulfonamide ([VMIM][TFSI]). The RTIL may be 1-hydroxyundecanyl-3-methylimidazolium bis(trifluoromethane)sulfonamide ([(C₁₁OH)MIM][TFSI]). The RTIL may be 1-ethyl-3-methylimidazolium tricyanomethanide ([EMIM][TCM]). The RTIL may be tetrabutylphosphonium taurinate. The RTIL may be ([P4444][Tau]). The RTIL may be 1-ethyl-3-methylimidazolium dicyanamide ([EMIM][DCA]). The RTIL may be 1-(2,3-dihydroxypropyl)-alkyl imidazolium dicyanamide ([(dhp)MIM][DCA]). The RTIL may be 1-(2,3-dihydroxypropyl)-3-alkyl imidazolium tetrafluoroborate ([(dhp)MIM][BF4]). The RTIL may be 1-(2,3-dihydroxypropyl)-3-alkyl imidazolium bis(trifluoromethane)sulfonimide ([(dhp)MIM][TFSI]). The RTIL may also be 1-(2,3-dihydroxypropyl)-3-alkyl imidazolium hexafluorophosphate ([(dhp)MIM][PF6]). These exemplary RTILs are further illustrated below at Table 1.

TABLE 1
Exemplary RTILs.

| Abbreviation | Chemical Name | Structure |
|---|---|---|
| [EMIM][TSFI] | 1-ethyl-3-methylimidazolium bis(trifluoromethane)sulfonimide | 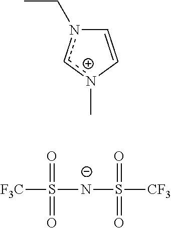 |
| [VEIM][TSFI] | 1-vinyl-3-ethyl-imidazolium bis(trifluoromethane)sulfonimide | 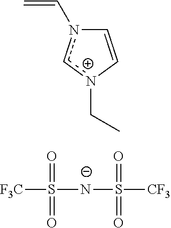 |
| [HMIM][TSFI] | 1-hexyl-3-methyl-imidazolium bis(trifluoromethane)sulfonimide | 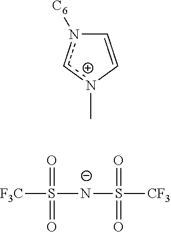 |
| [AMIM][TSFI] | 1-allyl-3-methyl-imidazolium bis(trifluoromethane)sulfonimide | 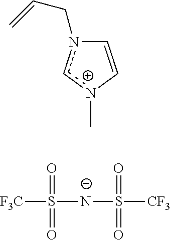 |
| [HBIM][TSFI] | 1-hexyl-3-butyl-imidazolium bis(trifluoromethane)sulfonimide | 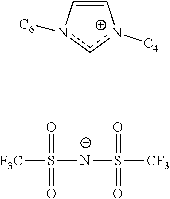 |
| [VMIM][TSFI] | 1-vinyl-3-methylimidazolium bis(trifluoromethane)sulfonimide | 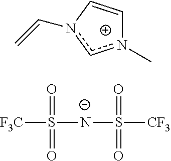 |
| [(C₁₁OH)MIM] [TSFI] | 1-hydroxyundecanyl- 3-methylimidazolium bis(trifluoromethane)sulfonimide | 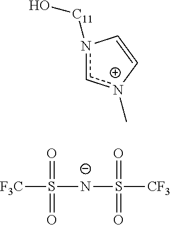 |
| [EMIM][TCM] | 1-ethyl-3-methylimidazolium tricyanomethanide | 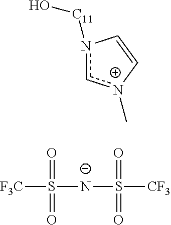 |
| [P4444][Tau] | tetrabutylphosphonium taurinate | 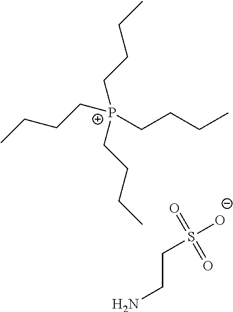 [SMILES: CCCC[P+](CCCC)(CCCC)CCCC ; NCCS(=O)(=O)[O-]] |
| [EMIM][DCA] | 1-ethyl-3-methylimidazolium dicyanamide | 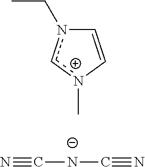 |
| [DMIM][Tf2N] or [DEIM][Tf2N] | 1-(2,3-dihydroxypropyl)-3- methylimidazolium bis(trifluoromethanesulfonimide) or 1-(2,3-dihydroxypropyl)- 3-ethylimidazolium bis(trifluoromethanesulfonimide) | 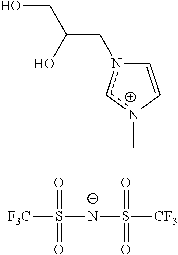 |
| [DMIM][BF4] or [DEIM][BF4] | 1-(2,3-dihydroxypropyl)-3- methylimidazolium tetrafluoroborate or 1-(2,3-dihydroxypropyl)- 3-ethylimidazolium tetrafluoroborate | 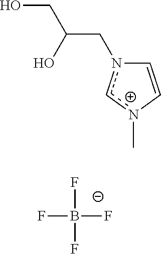 |
| [DMIM][DCA] or [DEIM][DCA] | 1-(2,3-dihydroxypropyl)-3- methylimidazolium dicyanamide or 1-(2,3-dihydroxypropyl)- 3-ethylimidazolium dicyanamide | 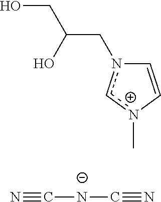 |
| [DMIM][PF6] or [DEIM][PF6] | 1-(2,3-dihydroxypropyl)-3- methylimidazolium hexafluorophosphate or 1-(2,3-dihydroxypropyl)- 3-ethylimidazolium hexafluorophosphate | 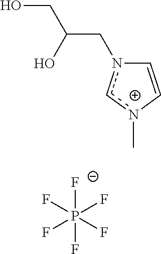 |

The RTIL may be [Rmim][TSFI]. In particular, the RTIL may be [Rmim][TSFI], wherein R is ethyl; that is, the RTIL may be 1-ethyl-3-methylimidazolium bis(trifluoromethanesulfonyl)imide ([EMIM][TSFI]).

The liquid medium may be a mixture of an aqueous medium and an RTIL. For such mixtures, the volume ratio may be between about 99:1 and about 1:99 aqueous medium/RTIL, such as between about 99:1 and about 95:5 aqueous medium/RTIL, between about 95:5 and about 90:10 aqueous medium/RTIL, between about 90:10 and about 85:15 aqueous medium/RTIL, between about 85:15 and about 80:20 aqueous medium/RTIL, between about 80:20 and about 75:25 aqueous medium/RTIL, between about 75:25 and about 70:30 aqueous medium/RTIL, between about 70:30 and about 65:35 aqueous medium/RTIL, between about 65:35 and about 60:40 aqueous medium/RTIL, between about 60:40 and about 55:45 aqueous medium/RTIL, between about 55:45 and about 50:50 aqueous medium/RTIL, between about 50:50 and about 55:45 aqueous medium/RTIL, between about 55:45 and about 45:65 aqueous medium/RTIL, between about 45:65 and about 40:60 aqueous medium/RTIL, between about 40:60 and about 35:65 aqueous medium/RTIL, between about 35:65 and about 30:70 aqueous medium/RTIL, between about 30:70 and about 25:75 aqueous medium/RTIL, between about 25:75 and about 20:80 aqueous medium/RTIL, between about 20:80 and about 15:85 aqueous medium/RTIL, between about 15:85 and about 10:90 aqueous medium/RTIL, between about 10:90 and about 5:95 aqueous medium/RTIL, or between about 5:95 and about 1:99 aqueous medium/RTIL. In particular, the molar ratio may between about 70:30 and about 20:80 aqueous medium/RTIL, between about 60:40 and about 30:70 aqueous medium/RTIL, or at about 40:60 aqueous medium/RTIL.

In general, the liquid electrolyte medium may further comprise one or more non-aqueous solvents. The one or more non-aqueous solvents may be a polar protic solvent, a polar aprotic solvent, a non-polar solvent, or combinations thereof. Suitable examples of polar protic solvents include but are not limited to alcohols such as methanol, ethanol, isopropanol, n-propanol, iso-butanol, n-butanol, s-butanol, t-butanol, and the like; diols such as propylene glycol; organic acids such as formic acid, acetic acid, and so forth; amines such as trimethylamine, or triethylamine, and the like; amides such as formamide, acetamide, and so forth; and combinations of any of the above. Non-limiting examples of suitable polar aprotic solvents include acetonitrile, dichloromethane (DCM), diethoxymethane, N,N-dimethylacetamide (DMAC), N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, 1,4-dioxane, N-methyl-2-pyrrolidinone (NMP), ethyl formate, formamide, hexamethylphosphoramide, N-methylacetamide, N-methylformamide, methylene chloride, nitrobenzene, nitromethane, propionitrile, sulfolane, tetramethylurea, tetrahydrofuran (THF), 2-methyltetrahydrofuran, trichloromethane, and combinations thereof. Suitable examples of non-polar solvents include, but are not limited to, alkane and substituted alkane solvents (including cycloalkanes), aromatic hydrocarbons, esters, ethers, combinations thereof, and the like. Specific non-polar solvents that may be employed include, for example, benzene, butyl acetate, t-butyl methyl ether, chlorobenzene, chloroform, chloromethane, cyclohexane, dichloromethane, dichloroethane, diethyl ether, ethyl acetate, diethylene glycol, fluorobenzene, heptane, hexane, isopropyl acetate, methyl tetrahydrofuran, pentyl acetate, n-propyl acetate, tetrahydrofuran, toluene, and combinations thereof.

In general, the TPE gel polymer electrolyte composite may have a liquid content ranging from 0 w/v % to about 95 w/v %. In various embodiments, the TPE gel polymer electrolyte may have a liquid content ranging from 0 w/v % to about 85 w/v %, from 0 w/v % to about 5 w/v %, from about 5 w/v % to about 10 w/v %, from about 10 w/v % to about 15 w/v %, from about 15 w/v % to about 20 w/v %, from about 20 w/v % to about 25 w/v %, from about 25 w/v % to about 30 w/v %, from about 30 w/v % to about 35 w/v %, from about 35 w/v % to about 40 w/v %, from about 40 w/v % to about 45 w/v %, from about 45 w/v % to about 50 w/v %, from about 50 w/v % to about 55 w/v %, from about 55 w/v % to about 60 w/v %, from about 65 w/v % to about 70 w/v %, from about 70 w/v % to about 75 w/v %, from about 75 w/v % to about 80 w/v %, from about 80 w/v % to about 85 w/v %, from about 85 w/v % to about 90 w/v %, or from about 90 w/v % to about 95 w/v %.

The TPE gel polymer electrolyte composites have some unique and unexpected properties. The TPE gel polymer electrolyte composites have a lubricious surface meaning the surface is smooth, glassy in appearance, and slippery with a low coefficient of friction.

Generally, the TPE gel polymer electrolytes have a bulk modulus averaged over the initial 10% strain of about 0.1 megapascals (MPa) to about 25 MPa. In various embodiments, the TPE gel polymer electrolytes have a bulk modulus averaged over the initial 10% strain of about 0.1 megapascals (MPa) to about 25 MPa. In various embodiments, the TPE gel polymer electrolyte composites have a modulus of about 0.1 MPa to about 1 MPa, from about 1 MPa to about 2 MPa, from about 2 MPa to about 3 MPa, from about 3 MPa to about 4 MPa, from about 4 MPa to about 5 MPa, from about 5 MPa to about 6 MPa, from about 6 MPa to about 7 MPa, from about 7 MPa to about 8 MPa, from about 8 MPa to about 9 MPa, from about 9 MPa to about 10 MPa, from about 10 MPa to about 11 MPa, from about 11 MPa to about 12 MPa, from about 12 MPa to about 13 MPa, from about 13 MPa to about 14 MPa, from about 14 MPa to about 15 MPa, from about 15 MPa to about 16 MPa, from about 16 MPa to about 17 MPa, from about 17 MPa to about 18 MPa, from about 18 MPa to about 19 MPa, from about 19 MPa to about 20 MPa, from about 20 MPa to about 21 MPa, from about 21 MPa to about 22 MPa, from about 22 MPa to about 23 MPa, from about 23 MPa to about 24 MPa, or from about 24 MPa to about 25 MPa.

In general, the TPE gel polymer electrolyte composites have a toughness of about 1 MJ/m³ to about 120 MJ/m³. In various embodiments, the TPE gel polymer electrolyte composites have a toughness of about 1 MJ/m³ to about 5 MJ/m³, from about 5 MJ/m³ to about 10 MJ/m³, from about 10 MJ/m³ to about 20 MJ/m³, from about 20 MJ/m³ to about 30 MJ/m³, from about 30 MJ/m³ to about 40 MJ/m³, from about 40 MJ/m³ to about 50 MJ/m³, from about 50 MJ/m³ to about 60 MJ/m³, from about 60 MJ/m³ to about 70 MJ/m³, from about 70 MJ/m³ to about 80 MJ/m³, from about 80 MJ/m³ to about 90 MJ/m³, from about 90 MJ/m³ to about 100 MJ/m³, from about 100 MJ/m³ to about 110 MJ/m³, or from about 110 MJ/m³ to about 120 MJ/m³.

With the above properties, the TPE gel polymer electrolyte composites are resistant to fatigue, wear, fracture, degradation, or any combination thereof.

In one embodiment, the TPE gel polymer electrolyte composite may have a fatigue resistance to at least 500,000 compression cycles, such as at least 600,000 compression cycles, such as at least 700,000 compression cycles, such as at least 800,000 compression cycles, such as at least 900,000 compression cycles, such as at least 1,000,000 compression cycles, such as at least 1,500,000 compression cycles, such as at least 2,000,000 compression cycles, such as at least 2,500,000 compression cycles, such as at least 3,000,000 compression cycles, such as at least 3,500,000 compression cycles, such as at least 4,000,000 compression cycles, such as at least 4,500,000 compression cycles, such as at least 5,000,000 compression cycles, or such as at least 10,000,000 compression cycles. In counting the number of compression cycles, the cycles are preferably continuous, but need not be so, having a resting period between shorter runs of cycles.

The compression cycles may operate with at least 30% compression at a frequency of about 2 Hz. The fatigue resistance is characterized by a modulus recoverable to at least 80% of its value before the compression cycles were run, such as to at least 90%, to at least 95% or to at least 99% of its value before the compression cycles were run.

The TPE gel polymer electrolyte composites have a unique and unexpected property. The ionic conductivity of the gel polymer electrolyte hydrogel is comparable to the liquid electrolyte medium. In some embodiments, the ionic conductivity is equal to or higher than the liquid electrolyte medium.

### VIII. Methods of Preparing the TPE Gel Polymer Electrolyte Composite

Another aspect of the present disclosure provides for methods of preparing the TPE gel polymer electrolyte composites. The method comprises contacting the at least two thermoplastic elastomers in a molar ratio from between 1:99 and 99:1 to form a TPE dry blend. The TPE dry blend is heated under conditions mechanical mixing, mechanical extrusion or mechanical pressure to form a TPE composite melt. The TPE composite melt is allowed to attain ambient temperature to form an TPE composite solid. The TPE composite solid is then contacted with a liquid medium to form a TPE gel polymer electrolyte composite.

The liquid medium may be a non-aqueous solvent, non-aqueous liquid electrolyte, or a combination thereof. For example, any liquid medium described herein may be used.

The TPE composite solid may be contacted with the liquid medium at a temperature above −10° C. and below about 160° C., such as above 0° C. and below about 50° C., or at about 25° C. The temperature may be between about −10° C. and about −5° C., between about −5° C. and about 0° C., between about 0° C. and about 5° C., between about 5° C. and about 10° C., between about 10° C. and about 15° C., between about 15° C. and about 20° C., between about 20° C. and about 25° C., between about 25° C. and about 30° C., between about 30° C. and about 35° C., between about 35° C. and about 40° C., between about 40° C. and about 45° C., between about 45° C. and about 50° C., between about 50° C. and about 55° C., between about 55° C. and about 60° C., between about 60° C. and about 65° C., or between about 65° C. and about 70° C., between about 70° C. and about 75° C., between about 75° C. and about 80° C., between about 80° C. and about 85° C., between about 85° C. and about 90° C., between about 90° C. and about 95° C., between about 95° C. and about 100° C., between about 100° C. and about 105° C., between about 105° C. and about 110° C., between about 110° C. and about 115° C., between about 115° C. and about 120° C., between about 120° C. and about 125° C., between about 125° C. and about 130° C., between about 130° C. and about 135° C., between about 135° C. and about 140° C., between about 140° C. and about 145° C., between about 145° C. and about 150° C., between about 150° C. and about 155° C., or between about 155° C. and about 160° C.

### IX. Applications

The TPE GPE composites may be used as adhesives, coating materials, elastic separation membranes such as for light gases, protein assemblies and biologics, and mechanical energy absorbers, such that found in footwear, sportswear, helmets and other protective gear, and sports equipment. The TPE GPE composites may also be used as separators in battery cells or fuel cells.

The TPE gel polymer electrolyte composites of the present disclosure exhibit excellent ionic conductivity making the TPE gel polymer electrolyte composites especially suited for battery separators and flexible ionic conductors.

### (a) Gas Separation

The TPE gel polymer electrolyte composites may be used as elastic separation membranes for light gases, such as mixtures of carbon dioxide (CO₂), methane (CH₄), ethane, propane, butane, water, oxygen (O₂), nitrogen and argon. The mixture of light gases may be crude natural gas (such as that produced at a natural gas well), flue gas, or atmosphere. In particular, CO₂ is emitted from coal-fired power plants in “flue gas,” which contains 10-15% CO₂ along with N₂ (70-80%), water, O₂, and other trace gases.

Existing technologies for the separation of CO₂ from flue gas include aqueous amine scrubbing, pressure swing absorption, and cryogenic distillation. Implementing these technologies requires about 30% of the energy produced by the power plant, making them economically unsustainable. Membrane-based alternatives are being investigated at the pilot plant scale as a superior solution for separating CO₂ from flue gas. Successful membrane technologies offer several advantages over traditional methods for lower operating energies, modular scalability, a reduced physical footprint, and elimination of volatile chemicals.

To successfully apply to flue gas separations, membranes must have high CO₂ permeance and reasonable CO₂/N₂ selectivities (>20:1), be processable into substantially defect-free thin films, have long operating lifetimes, and have reasonable production costs. The range of CO₂/N₂ selectivities can and will vary. Generally, the selectivity may be between about 20:1 and about 60:1, such as about 20:1 to about 25:1, about 25:1 to about 30:1, about 30:1 to about 35:1, about 35:1 to about 40:1, about 40:1 to about 45:1, about 45:1 to about 50:1, about 50:1 to about 55:1, or about 55:1 to about 60:1. The selectivity may be greater than about 20:1. The selectivity may be less than about 60:1.

New membrane materials may be screened by measuring single-gas permeability and selectivity, which are compared with performance values of existing materials using a comprehensive Robeson Plot, which are used in membrane science to gauge the performance of a membrane relative other materials as well to measure progress in a particular separation over time. Many other critical properties, such as mechanical stability over time, processability into free-standing or stable thin films, and compatibility with current module configurations, may also be addressed.

The CO₂/N₂ separation performance of the RTIL-TPE gel polymer electrolyte composite membranes disclosed herein was characterized by transmembrane pressure differentials exceeding about 400 kPa. RTIL-TPE gel polymer electrolyte composite membranes disclosed herein exhibit figures of merit pushing the limits of the 2008 Robeson plot upper bound, while maintaining exceptional mechanical integrity as a free-standing film, even in the swollen state. RTIL-TPE gel polymer electrolyte composite membranes disclosed herein exhibit unique tensile and compressive properties under cyclic loading conditions, and the extended CO₂/N₂ separation performance of these membranes greater than 28 days. No prior reports have been made of free-standing RTIL-TPE gel polymer electrolyte composite membranes prepared from melt-state processing of TPE composites swollen with RTIL and subsequently employed for light gas separations. Use of melt-state processing and self-assembly combined with diblock copolymer as a significant blend component, dramatically enhanced RTIL-TPE gel polymer electrolyte composite membranes to mechanically compete in CO₂/light gas separation.

### (b) Battery Separators

The TPE gel polymer electrolyte composites disclosed herein may also be used to make separators in battery cells or fuel cells. The battery separator is a critical component in liquid electrolyte batteries and is placed between the positive electrode and the negative electrode to prevent physical contact of the electrodes while enabling free ionic transport and isolating electronic flow. Generally, a battery separator is a microporous layer consisting of either a polymeric membrane or a non-woven fabric mat. The battery separators described herein are chemically and electrochemically stable towards the electrolyte and electrode materials under ordinary battery operation. These battery separators are also mechanically strong enough to withstand the high tension during the battery assembly operation.

Structurally, the battery separator has sufficient porosity to absorb liquid electrolyte for the high ionic conductivity. One of skill in the art would recognize that the battery separator adds electrical resistance and takes up space inside the battery, which can adversely affect battery performance. Therefore, selection of an appropriate separator is critical to the battery performance, including energy density, power density, cycle life and safety. The battery separators described herein satisfy these performance criteria. Especially for high energy and power densities, the battery separator must be very thin and highly porous while still remaining mechanically strong. For battery safety, the battery separator may shut the battery down if overheated, such as the occasional short circuit, so that thermal runaway can be avoided. The shutdown function can be obtained through a multilayer design of the battery separator, in which at least one layer melts to close the pores below the thermal runaway temperature and the other layer provides mechanical strength to prevent physical contact of the electrodes.

The function of a battery separator described herein is to prevent physical contact of the positive and negative electrodes while permitting free ion flow. The battery separator itself does not participate in any cell reactions, but its structure and properties considerably affect the battery performance, including the energy and power densities, cycle life, and safety.

The battery separator materials described herein, namely the TPE composites, are chemically stable against the electrolyte and electrode materials under ordinary battery operation, especially under the strongly reductive and oxidative environments when the battery is fully charged. Meanwhile, the battery separator does not degrade or lose mechanical strength during ordinary battery operation over the typical lifetime of a battery. A method for one of skill in the art to verify chemical stability is by calendar life testing.

The low thickness of the battery separators described herein permits high energy and power densities. Although a low thickness may adversely affect the mechanical strength and safety of the separator, the TPE gel polymer electrolyte composites are strong enough for this application. A thickness of 25.4 μm (1 mil) is the standard for consumer rechargeable batteries. As such, battery separators described herein may have a thickness between about 10 μm and about 40 μm, such as between about 10 μm and about 20 μm, between about 20 μm and about 30 μm, or between about 30 μm and about 40 μm. The battery separators may have a uniform thickness across the area of the separators, thereby aiding long cycle life of the batteries in which it is used. The thickness can be measured using the T411 om-83 method developed under the auspices of the Technical Association of the Pulp and Paper Industry.

The battery separators described herein may wet easily in the liquid medium and retain the liquid medium permanently (over the typical lifetime of a battery). The former facilitates the process of electrolyte filling in battery assembly and the latter increases cycle life of the battery. There is no generally accepted test for battery separator wettability. Placing a droplet of electrolyte on the separator and observing whether or not the droplet quickly wicks into the battery separator is an easy way to indicate sufficient wettability.

The battery separators lay flat and do not bow or skew when laid out and soaked with liquid medium. The battery separator remain stable in dimensions over a wide temperature range during the typical lifetime of a battery.

Most battery separator cost is in the manufacturing process. The process described herein is cost-effective, in that it reduces the battery separator cost. Many properties above are associated with each other and may be in a trade-off relationship. For example, reducing the separator thickness increases battery energy and power densities, but it may also lower the mechanical strength of the battery separator. In practical applications, one of skill in the art would understand to appropriately weight the requirements above among the performance, safety and cost.

As various changes could be made in the above-described methods without departing from the scope of the invention, it is intended that all matter contained in the above description and in the examples given below, shall be interpreted as illustrative and not in a limiting sense.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Examples 1-27

### Materials and Synthetic Methods

All reagents were purchased from Millipore-Sigma and all solvents were purchased from Fisher Chemical unless otherwise stated. Styrene monomer (99%, 50 ppm p-tert-butylcatechol inhibitor) was purified at 40° C. by distillations under static vacuum (15-30 mTorr) from di-n-butylmagnesium (1.0 M in heptane). Ethylene oxide (99.5+%, compressed gas) was purified by successive distillations from di-n-butylmagnesium (1.0 M in heptane) at 0-5° C. Tetrahydrofuran (THF) was sparged with argon (10 psi) for 45 min and then purified over two neutral alumina molecular sieve columns (Glass Contour, Inc.). Cyclohexane (CHX) was degassed with argon and purified through a neutral alumina column followed by a Q5 copper(II) oxide catalyst column (Glass Contour, Inc.). Other common chemicals and solvents were used as received unless otherwise stated. Ultra-high purity argon (99.998%, Airgas) was passed through a column of 5 Å molecular sieves and oxygen-absorbing purifier column (Matheson Trigas). Potassium naphthalenide (KNAP) solution was prepared by mixing solid potassium metal (98% chunks in mineral oil, rinsed in cyclohexane prior to use) and a slight excess of recrystallized naphthalene under Ar in an air-free schlenk graduated cylinder in dry THF.

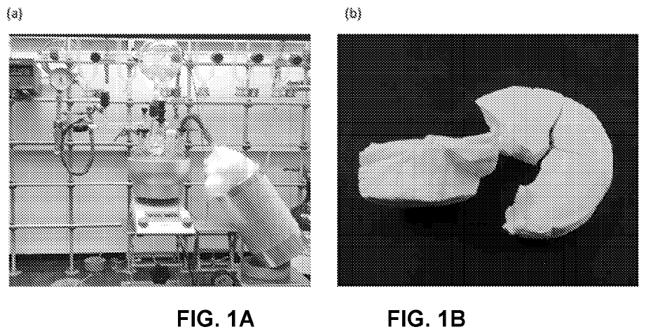

The synthetic apparatus is based on a multiport air-free glass reactor system that allows for the stepwise air-free addition of multiple monomer and initiators systems as shown in FIG. 1.

### Molecular and Material Characterization

### NMR, GPC, Thermal Analysis, and SAXS

¹H NMR spectra were collected in CDCl3 using a Bruker Advance NEO 400 MHz Spectrometer equipped with Prodigy BBFO cryo-probe (ns=256, 10 s delay for polymer samples). Gel permeation chromatography (GPC) was performed on a Viscotek RIMax system fitted with three 7.5×300 mm PolyPore (Agilent) columns in series, an Alltech external column oven set to 40° C., and a Viscotek differential refractive index (RI) detector with sample concentrations of 2 mg mL⁻¹. Stabilized THF running at 1.0 mL min⁻¹ was used as the eluent and run time was 45 minutes for all samples. Thermal analysis was performed on a TA Instruments TGA Q500 and Discovery DSC 2500.

Synchotron small angle x-ray scattering (SAXS) experiments were run at the 12-ID-B beamline at the Advanced Photon Source (Argonne, IL) for all neat polymer samples. All SAXS measurements utilized a beam energy of 13.30 keV (0.9322 Å) and were recorded by a Pilatus 2M detector (1475×1679 pixel resolution) at a sample-to-detector distance of 3.6 m. The scattering wave vector, q, was calibrated using a silver behenate standard (d=58.38 Å). Samples were thermally processed for 15 minutes prior to SAXS measurements to initiate phase separation and minimize air bubbles. Dry polymer discs were sandwiched between Kapton tape and mounted to a multi-sample DSC pan holder made for the multi-sample heated stage. The samples remained at ambient pressure and were ramped between 100° C. and 200° C., with exposure times of 1 s for all data collected. SAXS of ion gel samples were collected using an in-house Rigaku SMax3000 High Brilliance three-pinhole SAXS system outfitted with a MicroMax-007HFM rotating anode (Cu Kα), a Confocal Max-Flux Optic, a Gabriel-type multi-wire area detector (1024×1024 pixel resolution), and a Linkam thermal stage.

### Electronic Impedance Spectroscopy (EIS)

Impedance spectroscopy measurements were conducted using a 1287 Potentiostat/1260 FRA combination from Solartron in the 0.1 Hz-200 kHz range applying a 100 mV sinusoidal voltage. The samples, swollen gel disks, were heated under vacuum to 50° C. prior to the first cycle of EIS measurements and their impedance was measured periodically as the vacuum oven cooled to RT. The samples were heated to 80° C. under vacuum for the second cycle of measurements. The EIS measurements for all swollen gels were done using a reusable quick assembly split coin cell (20 mm I.D.) from MTI corporation. All EIS measurements for the RTIL were done using standard CR2032 SS316 coin cells using Viton gaskets and stainless-steel spacers and springs.

### Mechanical Characterization

### Unconfined Compression Testing

Unconfined compression testing was performed at room temperature on a rheometer (Ares, TA Instruments) fitted with an 8 mm upper and 25 mm lower parallel plate. Swollen gel samples (all larger than 8 mm diameter and approximately 2 mm thick) were sandwiched between the parallel plates with an initial compressive force of 0.10 to 0.15 N force (˜2 to 3 kPa of initial compressive stress) to ensure full contact with the parallel plates. Compression was applied at a strain rate of 10% s^{-′} up to 50% strain for 10 successive compression/decompression cycles.

Overall compressive modulus of each gel was determined by averaging compressive stress between 0 and 50% strain across all 10 cycles, and then calculating the slope of the averaged data. The three replicates were then averaged to determine mean compressive modulus per polymer sample.

### Uniaxial Tensile Testing (Strain-to-Break and Cyclical Fatigue)

Tensile testing was performed at room temperature on a tensile tester (Instron Model 4442 electromechanical universal testing system) fitted with pneumatic tensile grips (pressurized to 15-20 psig depending on the stiffness of the gel). Gels were swollen to approximately 10 mm×35 mm×1 mm rectangles, punched into a dog-bone shape (where the cross section of the narrow section was 3 mm×˜1 mm), and then mounted in the grips with an initial gauge length of ˜20 mm between grips (measured once mounted). A pre-stress of approximately 3 to 5 kPa was applied prior to initiation of the test to ensure that the sample was properly straightened. Gels were stretched at a strain rate of 2% s^{-′} for strain-to-break testing, and at a rate of 10% s^{-′} from 0% to 200% strain for cyclic tensile fatigue testing.

Young's modulus was determined for each sample/extension cycle by taking the slope of the linear regression of the initial 10 to 20% strain on the stress vs strain (kPa vs A-1) curve.

### Rheology

Frequency sweeps were performed on a TA Instruments Ares rheometer fitted with 25 mm upper and lower parallel plates. Swollen gel samples (all approximately 25 mm in diameter and approximately 1 mm thick) were sandwiched between the parallel plates with an initial z-axis strain of 10% (˜200 to 600 g normal force, depending on the stiffness of the gel) to ensure full contact with the parallel plates. Oscillatory strain was set between 0.3 and 0.6%, depending on the maximum strain of the linear viscoelastic regime of the gel, determined by strain sweeps performed at 1 Hz.

### Fracture Toughness Testing

“Pure shear” fracture testing^{26,27} was adopted to characterize the fracture toughness of three SOS40 gels. Specifically, samples with dimensions of 49 mm×49 mm×1 mm (width×height×thickness) were prepared by swelling polymer discs that were melt pressed in a 20 mm×20 mm×0.4 mm square stainless-steel mold. Each sample was clamped by two sets of wide rigid plates (100 mm×25 mm) on a mechanical testing machine (Instron 5965), leaving a long strip area for fracture testing with the following dimensions: width L0=48 mm, height H0=20 mm and thickness T0=1.0 mm. An initial crack with length C0=10 mm was introduced at the middle of the left edge of each sample by a razor blade. The top boundary of each sample was subjected to a vertical displacement A at a fixed velocity of A=0.2 mm/s, while the bottom boundary of the sample was fixed. The applied loading is quantified by the stretch ratio I=1+Δ/H0 with an effective strain rate of Δ⁻/H0=0.01 s⁻¹. During the experiment, a digital camera (Canon XF10) was used to image the crack propagation process. In addition to the fracture tests, an unnotched control sample with the same dimensions was stretched under the loading conditions until failure.

### Example 1: Synthesis of Polystyrene-OH (S—OH) Macroinitiator

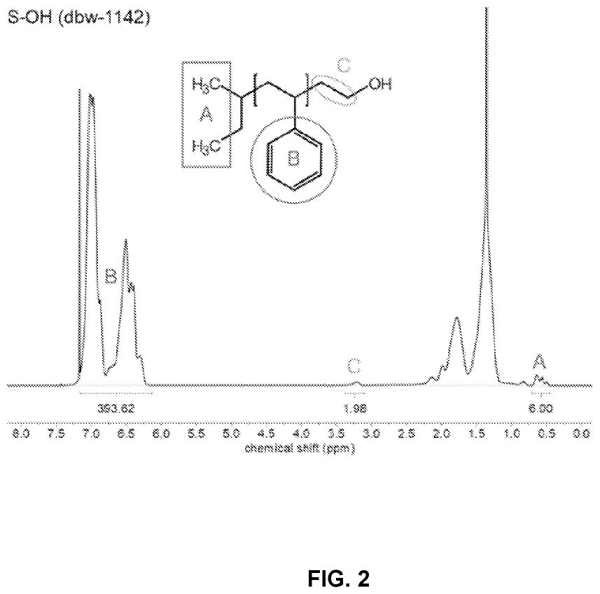

Synthesized of polystyrene-OH has been previously reported.^{19,25 1}H NMR characterization data is provided in FIG. 2.

### Example 2: Synthesis of Polystyrene-b-Poly(Ethylene Oxide)-H, (SO—H)

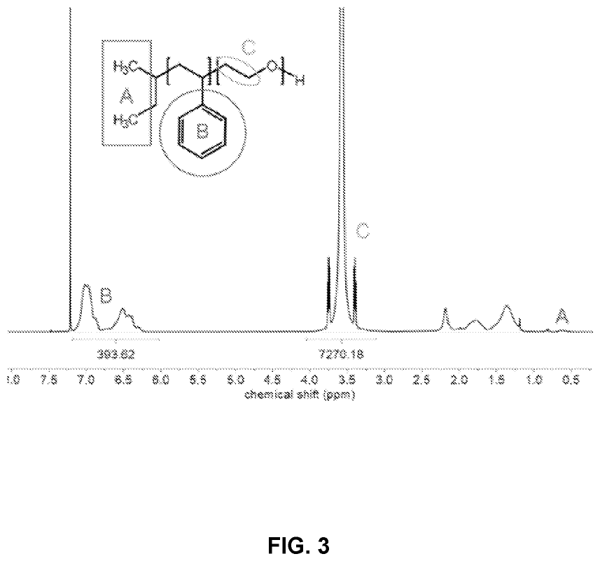

This procedure is a modification of synthetic methods originally published in 2010 by Guo and Bailey.¹⁹ A 2 L air-free reaction vessel fitted with glass stir bar, pressure gauge, transfer arm, and 1 L solvent bulb was filled with dry, unstabilized tetrahydrofuran (THF), was evacuated and backfilled with Ar gas five times. Under positive Ar pressure (1 psig), 1.729 g (0.216 mmol) of S—OH macroinitiator was added to the reactor. The reactor was evacuated and backfilled five more times and then evacuated overnight to ensure dryness of the S—OH. After backfilling with Ar (−3.5 psig), the THF was added to the reactor to dissolve the S—OH. The reactor was heated to 45° C. and then titrated (via 5 mL airtight glass Hamilton syringe) with concentrated KNAP until a light green color persisted in the reactor for approximately 20 minutes. After reducing the reactor pressure to −3.5 psig, 17.15 g (0.389 mol) of purified ethylene oxide monomer (EO, maintained at 0° C.) was added via air-free glass buret. The reaction was stirred for 24 hrs. The reaction was then allowed to cool to room temp, vented for 20 min, and terminated with −2 mL of 0.1 N HCl. The reaction was reduced to −0.6 L via rotary evaporator, precipitated into 4 L of pentane, and recovered using vacuum filtration. Finally, the product was fully dried under vacuum at ambient temperature for two days. Yield (SO—H)=16.83 g, 89.1% yield, FIG. 3 shows the ¹H NMR spectra.

### Example 3: Synthesis of a “One-Pot” Polymerization of Polystyrene-b-Poly(Ethylene Oxide)-b-Polystyrene (SOS83)

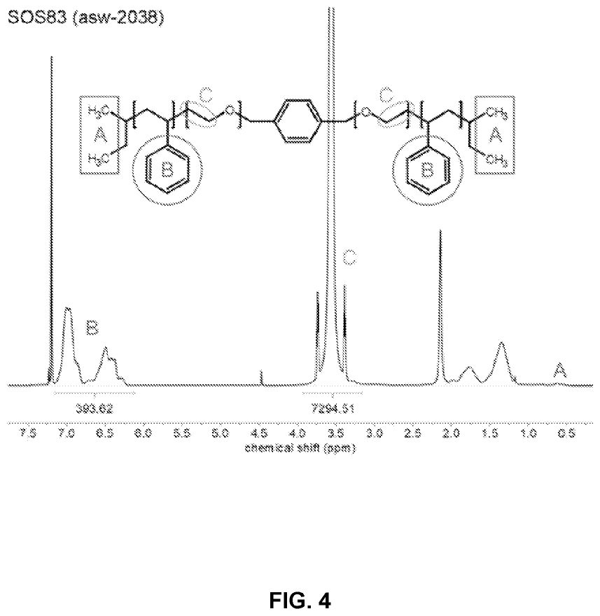

This synthesis proceeded similarly to the procedure for SO—H above, with the following changes: 1.41 g of S—OH macroinitiator was placed in the reactor and reacted with 14.1 g of purified ethylene oxide (EO) monomer. After stirring the reaction for 24 hours, the reactor was cooled for 1 hour prior to venting reactor with a needle and positive Ar pressure in order to remove unreacted EO without exposing reactor to air. The reactor was sealed again and re-titrated with a fresh solution of concentrated KNAP using a glass syringe. 0.547 g (2.07 mmol) recrystallized α,α′-dibromo-p-xylene (DBX, Tokyo Chemical Industry Co., Ltd.) was dried in a 100 mL purification flask for 20 min. Dry THF was added to the purification flask via cannula and the solution was weighed so the concentration was known. The total amount of KNAP required to titrate the reaction (titration #1 and #2) was used to calculate the amount of DBX (0.5 equivalents per K) to add to the reaction. 5.684 mL of DBX solution (0.226 mmol DBX, 0.55 equivalents) were added to the reactor via syringe pump over 10 hours. The reaction was reduced to 500 mL with rotary evaporation, precipitated into 3.5 L of pentane, and recovered using vacuum filtration. Finally, the product (83 wt % SOS triblock, 17 wt % SO diblock) was fully dried under vacuum at ambient temperature for two days. Yield (SOS83)=14.444 g, 93.0% yield. FIG. 4 shows the ¹H NMR spectra.

### Example 4: Synthesis of a “One-Pot” Polymerization of Polystyrene-b-Poly(Ethylene Oxide)-b-Polystyrene (ASW-2066)

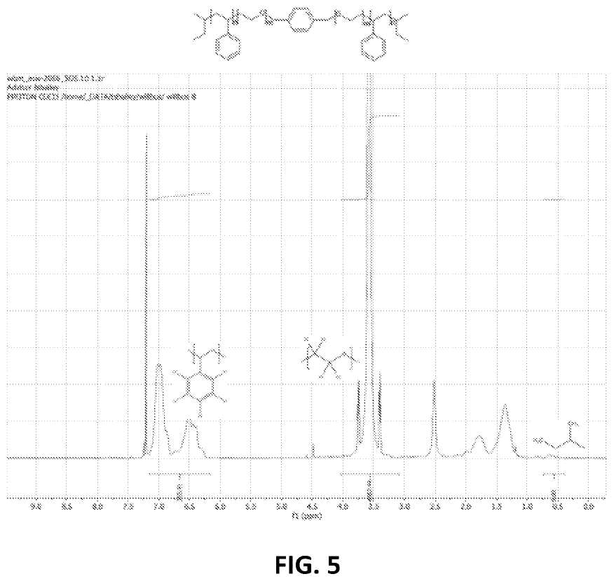

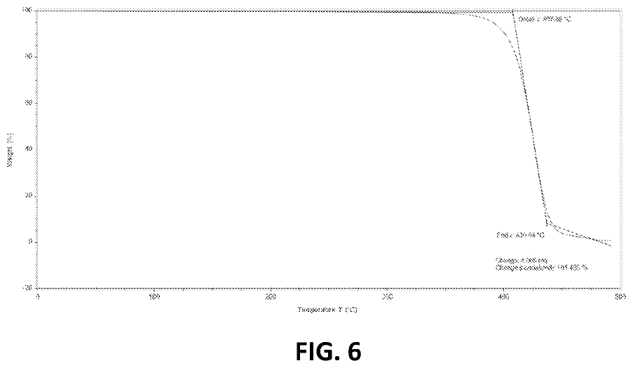

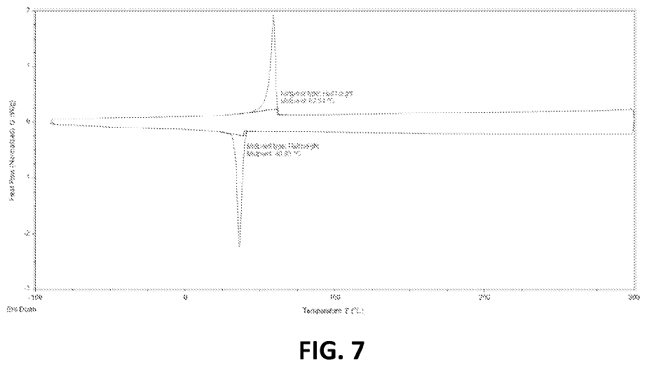

A 2 L air-free reaction vessel fitted with glass stir bar, pressure gauge, transfer arm, and 1 L solvent bulb filled with dry, unstabilized THF, was evacuated and backfilled with Ar gas five times. Under positive Ar pressure (1 psig), 1.56 g (0.195 mmol) of PS—OH macroinitiator was added to the reactor. The reactor was evacuated and backfilled five more times and then evacuated overnight to ensure dryness of the S—OH. After backfilling with Ar (˜3.5 psig), the THF was added to the reactor to dissolve the S—OH. The reactor was heated to 45° C. and then titrated (via 5 mL airtight glass Hamilton syringe) with concentrated KNAP until a light green color persisted in the reactor for approximately 20 minutes. After reducing the reactor pressure to ˜3.5 psig, 15.3 g (0.347 mol) of purified ethylene oxide monomer (EO, kept at 0° C.) was added via air-free glass buret. The reaction was stirred for 24 hrs. The reactor was then cooled for 1 hour prior to venting reactor with a needle and positive Ar pressure to remove unreacted EO without exposing reactor to air. The reactor was sealed again and re-titrated with a fresh solution of concentrated KNAP using a glass syringe. 0.858 g (3.25 mmol) recrystallized α,α′-dibromo-p-xylene (DBX, Tokyo Chemical Industry Co., Ltd.) was dried in a 100 mL purification flask for 20 min. Dry THF was added to the purification flask via cannula and the solution was weighed so the concentration was known. The total amount of KNAP required to titrate the reaction (titration #1 and #2) was used to calculate the amount of DBX (0.5 equivalents per K) to add to the reaction. 5.23 mL of DBX solution (0.325 mmol DBX) were added to the reactor via syringe pump over 10 hours. The reaction was reduced to 500 mL with rotary evaporation, precipitated into 3.5 L of pentane, and recovered using vacuum filtration. Finally, the product was fully dried under vacuum at ambient temperature for two days. FIG. 5 shows the ¹H NMR spectrum. FIG. 6 shows a thermal gravimetric analysis (TGA) spectrum. FIG. 7 shows a differential scanning calorimetry (DSC) spectrum.

### Example 6: Characterization of a Polystyrene-b-Polybutadiene-b-Polystyrene (SBS) TPE

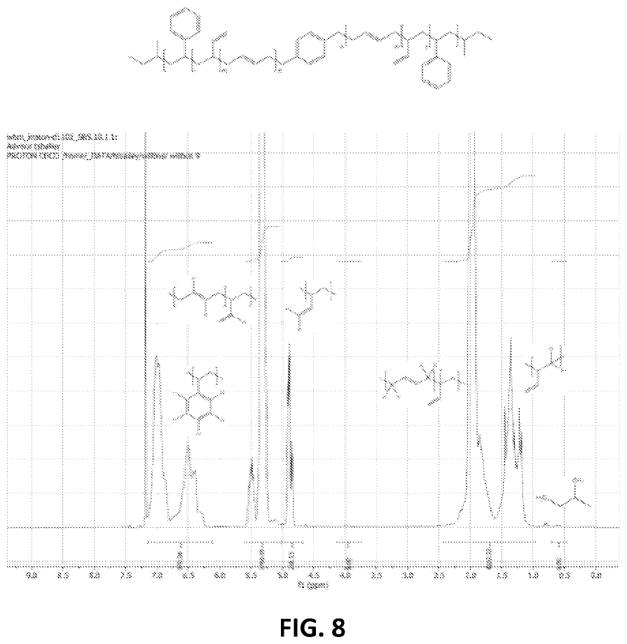

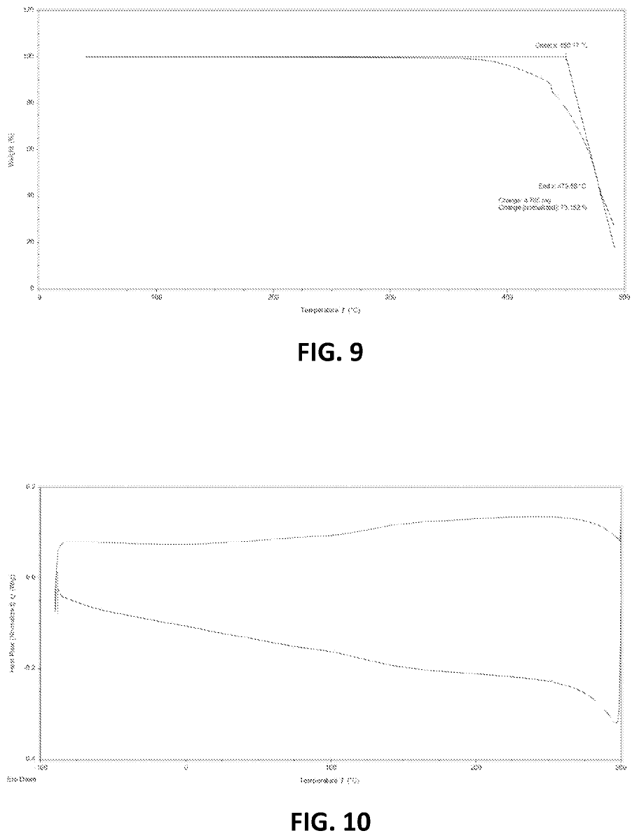

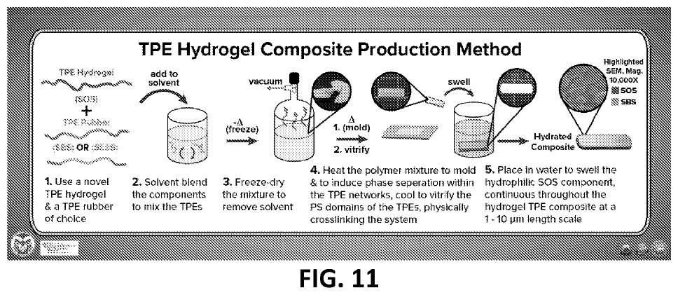

The characterization of the SBS TPE was accomplished using ¹H NMR, thermal gravimetric analysis (TGA), and differential scanning calorimetry (DSC). FIG. 8 shows the ¹H NMR spectrum. FIG. 9 shows a thermal gravimetric analysis (TGA) spectrum. FIG. 10 shows a differential scanning calorimetry (DSC) spectrum.

### Example 7: Method of Formation of a Thermoplastic Elastomer Dry Blend Through Dissolving in a Solvent and Evaporating the Solvent

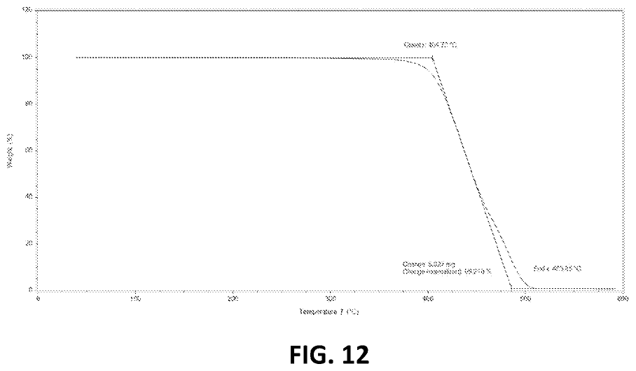

TPE dry blends were made by blending the TPE elastomers together with benzene as a co-solvent. Benzene was chosen for its low enthalpy of sublimation, making it desirable to use as a freeze-drying co-solvent. The benzene (50 ml) and the TPE components (1 g each) were added to a vessel that could be closed to air and attached to a vacuum line. While the vessel was open to air without being attached to the vacuum, the solution was stirred on a stir plate and heated with a heat gun, in intervals lasting 10 seconds or less, occurring about once every fifteen minutes. Mixing proceeded until the polymers were fully dissolved in solution, stirring for about 2 hours total. Once the components were well incorporated, the solution was then frozen by submerging in liquid nitrogen for about 15 minutes, vitrifying the mixture. To sublimate the benzene, the vessel was connected to the vacuum line and pumped down to a pressure of 15-30 mTorr. When the baseline pressure was reached, the liquid nitrogen was removed, and the benzene was sublimated out of the TPE blend. The TPE blend was then left under vacuum overnight to remove all the benzene. Once the TPE blend was dried, it was a light and fluffy powder and was placed in a freezer at 2° C. to be stored for future use. Once solvent blending was complete the TPE dry blend was ready to be melt processed, as shown in FIG. 11.

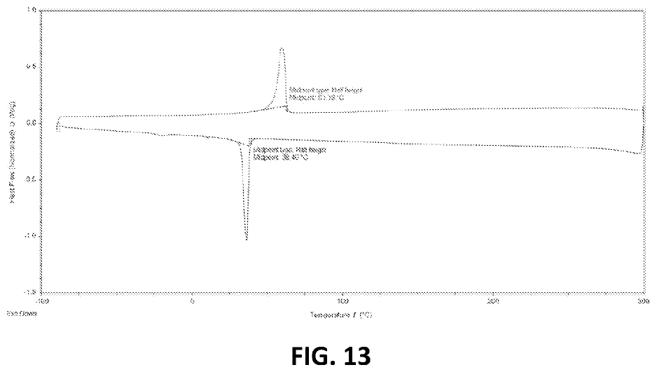

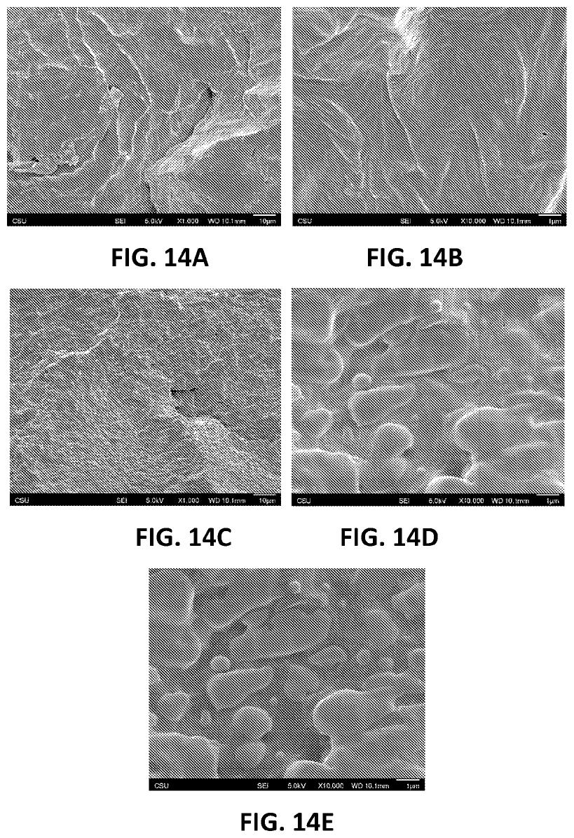

In one example, a TPE material comprised of polystyrene-polybutadiene-polystyrene TPE (1.0 g) was contacted with ASW-2066 SOS triblock copolymer (1.0 g) in about 50.0 ml of benzene. The resulting TPE dry blend was analyzed by thermal gravimetric analysis (TGA) (FIG. 12), and differential scanning calorimetry (DSC) (FIG. 13).

### Example 8: Method of Preparing a TPE Composite Using Heat and Pressure

In one embodiment, TPE dry blends were thermally processed using a Carver Model CH manual hydraulic press and stainless-steel rectangular molds (26 mm×7.5 mm×0.5 mm or 17 mm×6 mm×0.5 mm) or 3D printed molds for complex geometries. Samples were packed (overfilled by 50% more mass than required) into the mold that was placed on FEP coated Kapton FN (Dupont, 500FN131). Another sheet of Kapton was added on top of the mold and everything was placed between pre-heated aluminum plates in the melt press. The mold was heated to a temperature of 130° C. with slight pressure for 5 minutes. Then a pressure of 10,000 lbf was applied to a TPE dry blend containing 50% ASW-2006 SOS TPE and 50% SBS TPE rubber (D-1102) for 2 minutes to remove any trapped air bubbles in the sample. Samples were taken out of the melt press and allowed to cool to room temperature before being removed from the molds. If bubbles were still present in the sample after removing from the melt press, the sample was placed back and a greater pressure of 15,000 lbf was applied for another two minutes. Samples ranged from 7 to 11 minutes in the melt press to remove all bubbles.

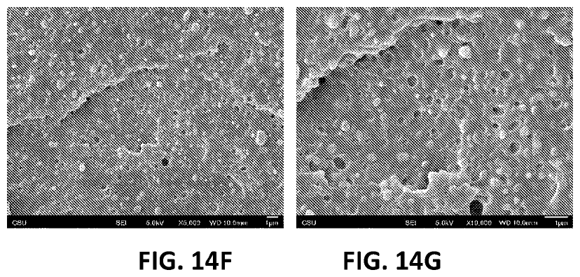

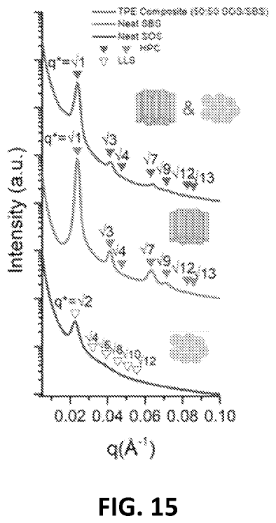

During melt processing, SOS TPE and the SBS TPE comprising the TPE dry blend were heated above their thermoformable temperatures and allowed to microphase separate into respective BCP domains. The PS blocks, that exist on the ends of all the polymer chains in the system, phase separate into like domains and vitrify after cooling to room temperature. Some of the PS blocks are close to another PS block from the other TPE component and cross the composite interface to arrange into a shared domain. When vitrified, the shared domain structure acts as a physical interfacial bond between the PEO domain and the rubbery TPE domain, leaving a welded interface between the two materials. Due to the fine mixing of the components and limited time in the melt state, the chains have limited diffusion range which forms microscale particles of the two TPE components. This method of welding is cost and time efficient because it only requires heat for short periods of time. Scanning Electron Microscopy (SEM) images were used to characterize the microstructure of the TPE composite formed by the use of heat and pressure. FIG. 14A-FIG. 14E show the microstructure at a series of magnifications indicating domain sizes of the constituent TPEs on the order of 0.1 to 20 microns. Small angle X-ray scattering (SAXS) was also used to characterize the structure internal to the individual TPE domains. FIG. 15 provides the SAXS data characterizing the morphology of the self-assembled TPE domains.

### Example 9: Method of Preparing a TPE Composite Using Mechanical Mixing (Micro Compounding)

In another embodiment, TPE dry blends were thermally processed using a Thermo Scientific HAAKE MiniLab 3 Micro Compounder. A TPE dry blend containing 50% ASW-2006 SOS TPE and 50% SEBS TPE rubber (Kraton G-1650) was heated under mixing at 150° C. at a screw speed of 150 rpm for 10 minutes before being extruded into a cylindrical filament. Scanning Electron Microscopy (SEM) images were used to characterize the microstructure of the TPE composite formed by micro compounding. FIG. 14F and FIG. 14G show the microstructure at a series of magnifications indicating domain sizes of the constituent TPEs on the order of 0.1 to 20 microns.

### Example 10: Method of Contacting a TPE Composite Solid with DI Water to Form and TPE Hydrogel Composite

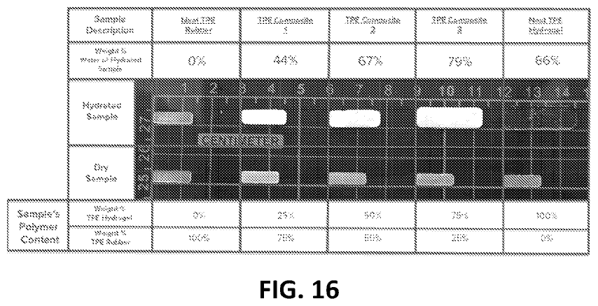

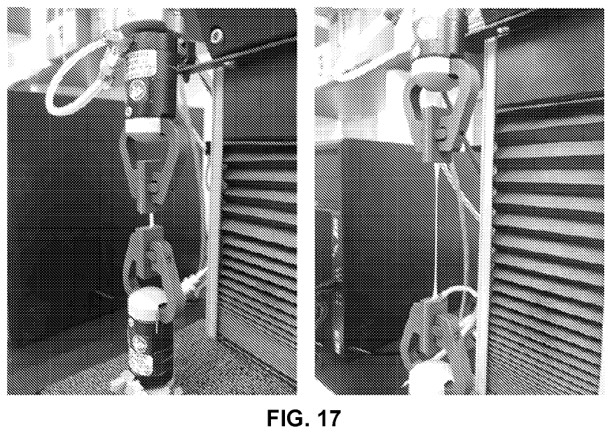

Melt processed TPE composites were weighed, and dimensions were measured. Once the dry samples were measured, they were individually placed into an excess of DI water and allowed to swell for 24 to 48 hours to reach swelling equilibrium. Prior to mechanical testing, the gels were dried on the surfaces using a Kimwipe to remove excess water. They were then weighed, and the dimensions were measured again and characterized according to the following formulae:

$$Water content [ wt . \% ] = \frac{{M}_{w} - {M}_{d}}{{M}_{w}} * 100 \% ,$$

$$Swelling Ratio ( Q ) = \frac{Mw - {M}_{d}}{Md} * 100 \% ,$$

Where M_{W} is the mass of the swollen gel and Mais the mass of the dry polymer composite. Example swelling characterization data and photographs of TPE hydrogel composites formed from 100% neat SBS (D-1102), 25% SOS 75% SBS, 50% SOS 50% SBS TPE, 75% SOS 25% SBS, and 100% neat SOS are given in FIG. 16.

### Example 11: Measuring the Tensile Properties of the TPE Hydrogel Composites

Mechanical testing samples were prepared by punching out the desired sample geometry from a swollen rectangular TPE hydrogel composite. Due to the variation in swelling capacity between sample types, all thicknesses were measured with digital calipers, immediately prior to testing.

Tensile test samples were punched with a dog-bone shaped punch custom made to be a small annex of an ASTM standard dog-bone geometry. Tensile testing was carried out using an Instron 4442 tensile testing instrument with a 5 N load cell, small pneumatic grips, and jaw faces covered in sandpaper. The large ends of the dog bone samples were loaded into pneumatic grips that closed at 90 psi. The sample was orientated in between the grips so the sample was vertical and not at an angle. The sample was placed under an initial stress of 1-10 kPa to ensure any slack was removed at the initiation of the test. The samples were extended at strain rate of 2% strain per second until break or slipping from the grips.

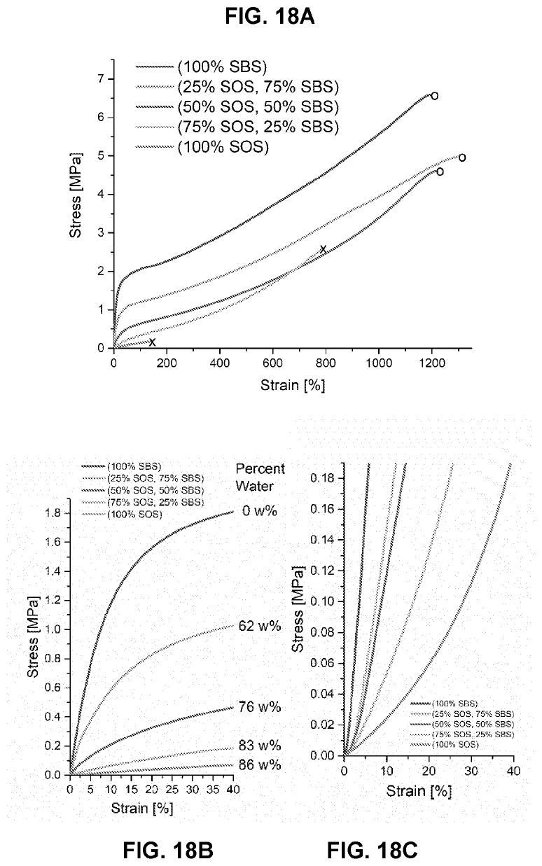

In one instance, TPE hydrogel composites based on TPE dry blends of ASW-2066 SOS TPE and SBS Kraton rubber (D-1102) were analyzed at compositions of the TPE dry blend of 100% neat SBS, 25% SOS 75% SBS, 50% SOS 50% SBS TPE, 75% SOS 25% SBS, and 100% neat SOS. FIG. 18A-FIG. 18B show the resulting tensile stress-strain behavior of the SOS/SBS (D-1102) TPE hydrogel composites described above. These TPE hydrogel composites with tensile moduli and bulk toughness values comparable to most thermoplastic elastomers, falling in the 10 to 40 MJ/m3 range (Table 6). Batch to batch reproducibility in both tensile and compressive behavior was high suggesting the manufacturing process at the lab scale is reproducible.

TABLE 6
Tensile Modulus and Toughness

| | | Young's | Initial | Bulk | |
|---|---|---|---|---|---|
| | | Modulus | Modulus | Modulus | |
| | | (Limit to | (Under | (Over 10% | |
| | | Zero) | 10% Strain) | Strain) | Toughness |
| Sample | % Water | [MPa] | [MPa] | [MPa] | [MJ/m\hat{ }3] |
| 100% SBS | 0% | 18.71 | 11.62 | 0.46 | 47.15* |
| 25% SOS, | 62% | 9.85 | 5.78 | 0.34 | 36.64* |
| 75% SBS | | | | | |
| 50% SOS, | 76% | 3.50 | 2.09 | 0.37 | 26.30* |
| 50% SBS | | | | | |
| 75% SOS, | 83% | 0.78 | 0.64 | 0.33 | 8.59 |
| 25% SBS | | | | | |
| 100% SOS | 86% | 0.19 | 0.21 | 0.12 | 0.14 |

The mechanical properties, modulus and toughness are enhanced by increased non-hydrogel elastomer (SBS) concentration. Percent water increases due to increased hydrogel elastomer (SOS) concentration in the material. *Toughness calculated at point of slip not fracture.

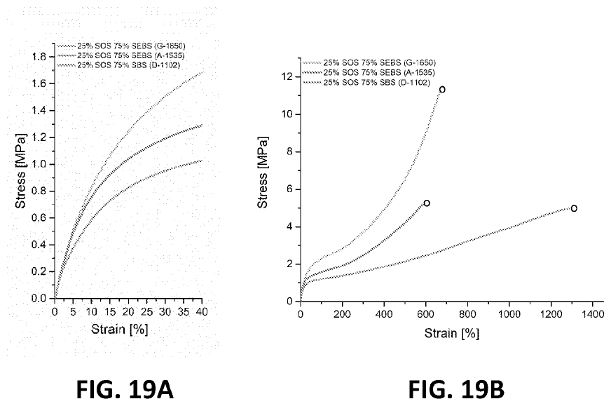

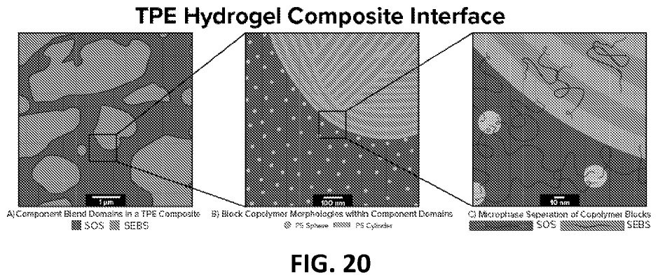

In another instance, TPE hydrogel composites based on TPE dry blends of ASW-2066 SOS TPE and SEBS Kraton rubber (G-1650 and A-1535) and were analyzed at compositions of the TPE dry blend of 25% SOS 75% SEBS. FIG. 19A and FIG. 19B show the resulting tensile stress-strain behavior of the SOS/SEES TPE hydrogel composites described above.

### Example 12: Measuring the Compressive Properties of the TPE Hydrogel Composites

Compression test samples were punched out using an 8 mm diameter circular punch. Unconfined compression testing was done in air on a TA Instruments Ares rheometer with parallel plates. While loading the samples for compression testing, swollen samples were sandwiched between upper and lower 25 mm diameter plates. Initially 10-20 g was applied to ensure full contact with the plates at the initiation of the test. Stress was applied to the samples at a strain rate of 2% per second until reaching a targeted maximum stress of 0.4 MPa for 6 successive loading/unloading cycles.

TPE hydrogel composites based on TPE dry blends of ASW-2066 SOS TPE and SBS Kraton rubber (D-1102) were analyzed at compositions of the TPE dry blend of 100% neat SBS, 25% SOS 75% SBS, 50% SOS 50% SBS TPE, 75% SOS 25% SBS, and 100% neat SOS. FIG. 18C shows the resulting compressive stress-strain behavior of the SOS/SBS (D-1102) TPE hydrogel composites described above.

### Example 13: Surgical Implantation of a Medial Meniscus Prototype into an Ovine Cadaver Knee

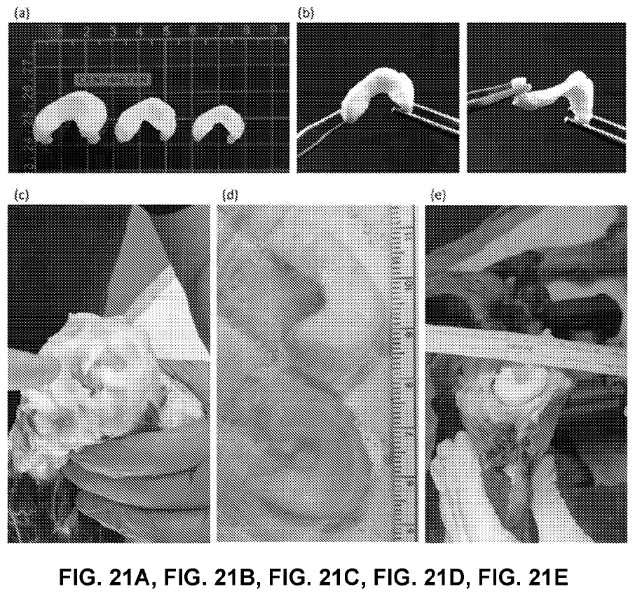

FIG. 21A, FIG. 21B, FIG. 21C, and FIG. 21D show TPE hydrogel composite. FIG. 21A shows the TPE hydrogel composite thermally molded into the shape of an ovine medial meniscus at three sizes, each containing different water contents. FIG. 21B shows twisting action demonstrating the elastic nature of the implant under load. FIG. 21C shows an intact ovine medial meniscus. FIG. 21D shows a side by side comparison of an actual ovine medial meniscus and a prototype implant. FIG. 21E shows the TPE hydrogel composite meniscal construct surgically implanted into the tibial plateau of a cadaver limb. All work done in collaboration with the CSU Preclinical Surgical Research Laboratory, CSU Veterinary School (Surgeon Jeremiah Easley).

### Example 14: Synthesis of 1-Ethyl-3-Methylimidazolium Bromide, [EMIM][Br]²⁴

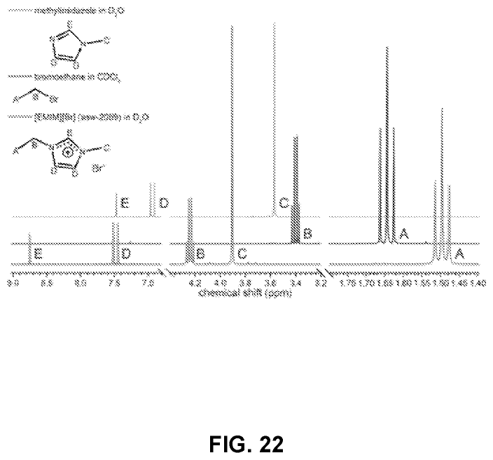

100 mL of 1-methylimidazole (1.26 mol, Alfa Aesar) and 100 mL of bromoethane (1.34 mol, Tokyo Chemical Industry Co., Ltd.) were added to a 1 L round bottom flask with PTFE stir bar, condenser, and argon bubbler. 200 mL of acetonitrile was then added to the flask. The mixture was stirred and heated to reflux at 40° C., slowly ramped to 75° C. over the course of two hours, and then left to stir overnight. The acetonitrile was removed via rotary evaporation and the product, [EMIM][Br] was precipitated into 600 mL of diethyl ether (Millipore). Twice the ether was decanted from the product and replaced with 500 mL of fresh ether and stirred. Finally, the ether was decanted from the product, and the product was vacuum dried overnight. Yield=218.8 g, 91.2% yield. FIG. 22 shows the ¹H NMR spectra.

### Example 15: Synthesis of 1-Ethyl-3-Methylimidazolium Bis(Trifluoromethylsulfonyl)Imide, [EMIM][TFSI]²⁴

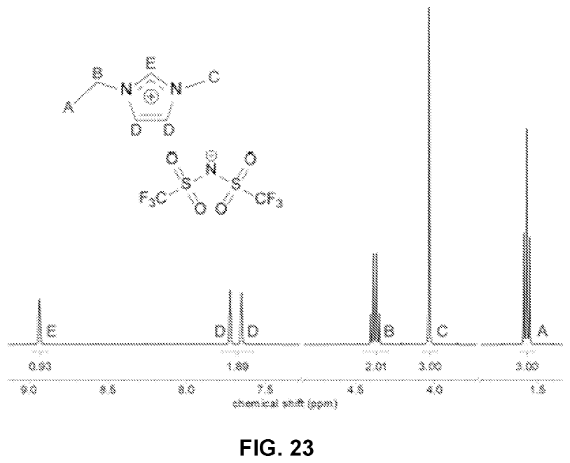

115 g (0.60 mol) of [EMIM][Br] was dissolved in 150 mL of deionized water (DI water) in a 500 mL round bottom flask with PTFE stir bar at room temperature. 190 g of Lithium bis(trifluoromethylsulfonyl)imide (0.66 mol, Tokyo Chemical Industry Co., Ltd.) were added to the flask and stirred for 3 hours. The reaction separated into distinct layers and the top water layer was decanted off. In order to remove the remaining LiBr salt produced from the reaction, the remaining reaction mixture was poured into a 1 L separatory funnel, along with 200 mL each of methylene chloride and DI water. After shaking, the water layer was removed and replaced with fresh water. This process was repeated two additional times. The methylene chloride layer containing ionic liquid was dried on a rotary evaporator and then further dried overnight on a Schlenk line with stirring at ambient temperature. Yield=227.124 g, 96.4% yield. FIG. 23 shows the ¹H NMR spectra.

### Example 16: Preparation of SOS Blends Via Precipitation

SOS blends were prepared by combining 0.540 g SOS83 with 0.559 g SO—H for SOS40 (40 wt % SOS triblock and 60 wt % SO diblock in the final blend) and 0.770 g SOS83 with 0.330 g SO—H for SOS57 (57 wt % SOS triblock and 43 wt % SO diblock). Each polymer mixture was then dissolved in 25 mL of chloroform and precipitated into 250 mL of pentane for a yield of approximately 1 g of each blend. The blends were dried overnight under vacuum.

### Example 17: Preparation of SOS40 Blend Via Freeze-Drying

2.400 g of SOS83 was combined with 2.484 g SO—H and 24.4 mg BHT (0.5 wt %) in ˜150 mL of benzene with stir bar in a flat-bottomed vacuum flask and sealed under ambient pressure. The mixture was stirred and very briefly heated until dissolved, then frozen on liquid N₂ (LN2) until solid (about 20-30 min). Once frozen, the flask was evacuated to ˜100 mTorr, at which point the LN2 flask was removed. The benzene was removed by vacuum over approximately 12 hours, during which the pressure in the flask slowly increased to ˜1 Torr and then gradually decreased back to baseline.

### Example 18: TPE Gel Polymer Electrolyte Fabrication: Thermal Processing and Swelling

Polymer samples were thermally processed using a Carver Model CH manual hydraulic press and various stainless-steel circular and rectangular molds as shown in Table 1.

TABLE 1
Summary of the dimensions of unswollen samples (size of the mold
used) and the average dimensions of the samples post-swelling

| | mold dimensions (mm) | | | average swollen dimensions (mm) | | |
|---|---|---|---|---|---|---|
| rectangular | width | length | thickness | width | length | thickness |
| tensile (SOS40) | 5 | 15 | 0.4 | 11.7 | 35.2 | 1.0 |
| tensile (SOS57) | 5 | 16 | 0.5 | 10.7 | 34.1 | 1.2 |
| tensile (SOS83) | 6 | 17 | 0.5 | 11.4 | 32.5 | 1.0 |
| fracture (SOS40) | 20 | 20 | 0.4 | 49 | 49 | 1.0 |
| circular | diameter | area (mm²) | thickness | diameter | area (mm²) | thickness |
| compression (SOS40) | 5.6 | 24 | 1 | 12.4 | 121 | 2.0 |
| compression (SOS57) | | | | 11.9 | 111 | 1.9 |
| compression (SOS83) | | | | 11.1 | 97 | 1.8 |
| rheology (SOS40) | 12 | 113 | 0.5 | 29 | 661 | 1.2 |
| rheology (SOS57) | | | | 27 | 573 | 1.1 |
| rheology (SOS83) | | | | 24 | 452 | 1.0 |

Samples were well packed (overfilled to at least 50% more mass than final disc) into the mold that was placed on a sheet of FEP coated Kapton FN (Dupont, 500FN131) on top of a pre-heated aluminum plate. The mold was then covered by a second piece of Kapton FN and topped with a second pre-heated aluminum plate. The mold was placed in the Carver press set to 150° C. and allowed to melt with slight pressure for 10 minutes. Pressure (˜5000 to 7000 lbs) was then applied to the sample for 5 minutes. Samples were removed and allowed to cool to room temperature before removing from molds.

The day prior to swelling, [EMIM][TFSI] ionic liquid was stirred, and vacuum dried on a Schlenk line overnight at ambient temperature. Melt processed discs were weighed and placed individually in an excess of dry [EMIM][TFSI] and allowed to swell for 12-48 hrs to achieve equilibrium swelling, depending on the thickness of the disc. Immediately before testing, the gels were removed from the ionic liquid, patted dry with Kim wipes, and weighed again.

### Example 19: Removal of Ionic Liquid from Swollen TPE GPE Composites for GPC Analysis

A small piece of each gel (˜100 mg) was trimmed off, placed in 7 mL of ethylene glycol, and stirred overnight to leech the ionic liquid from the gel. The resulting “dry” piece of polymer was removed from the ethylene glycol, rinsed briefly with acetone to remove excess ethylene glycol, and then dried in a vacuum oven.

### Example 20: Synthesis and Material Characterization

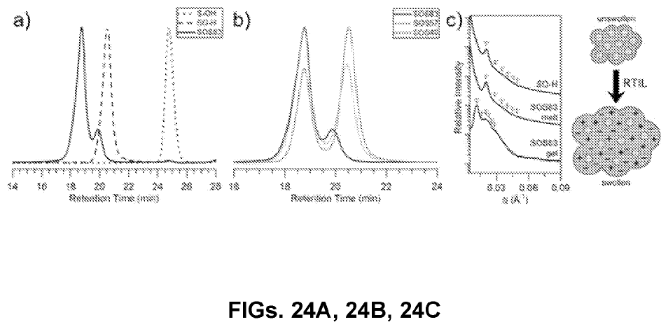

A “one-pot” anionic polymerization technique was conducted to grow narrowly dispersed, high molecular weight SO polymer (Table 2, FIG. 24A) and achieve high triblock coupling (83 wt % SOS) in a single, large reaction.

TABLE 2
List of Block Copolymers

| polymer | Mₙ (kDa) | Ð | f_{PS} |
|---|---|---|---|
| S—OH | 8.2 | 1.03 | 1.00 |
| SOS83 “one-pot” | 88.2 (diblock) | 1.04 | 0.101 |
| | 176.4 (triblock) | | |
| SO—H | 88.2 | 1.05 | 0.101 |

The product, SOS83, could then be used to make gels directly, or solution blended with a similar molecular weight SO—H diblock to reduce the amount of SOS triblock. Previously reported strategies for generating SOS triblock in a “one-pot” reaction,^{23,28} but the final triblock coupling was only ˜50% by mass and required time- and solvent-intensive fractionation to increase the amount of SOS triblock in the product in both cases. A simple addition of a second KNAP titration step, performed after the initial polymerization of ethylene oxide but before the addition of the coupling agent (DBX) was successful in increasing the yield of SOS triblock chains in the final product, such that fractionation was unnecessary. The second titration reactivates a reversibly dormant fraction of ethylene oxide chain ends produced during the extended reaction times required for the anionic polymerization of ethylene oxide. To demonstrate the full potential of these materials as polymer gel electrolyte elastomers three different triblock copolymer blend concentrations: high (83 wt % SOS), medium (57 wt % SOS) and low (40 wt % SOS) were selected (FIG. 24B). Because the amount of SOS triblock has been shown to directly affect swelling ratio and modulus in SOS TPE gel polymer electrolyte composites,^{19,22,23} this range of concentrations was selected to evaluate the impact of SOS triblock content on swelling ratio, ionic conductivity, and mechanical behavior when swollen in RTIL.

The observed form factor scattering in the melt state SAXS analysis (FIG. 24C) of both SOS83 and SO—H, indicative of a liquid-like packing of spherical domains that is typical for these materials at such high molecular weights and low fPS.^{19,21-23,25} When swollen, the morphology remained unchanged, however, the primary scattering wave vector (q*) of SOS83 was nearly halved (from 0.02012 Å⁻¹ to 0.01148 Å⁻¹), which translates to an almost two-fold increase in the domain spacing between spheres. This is consistent with our observations that the swollen dimensions of SOS83 gels are nearly double that of the thermally processed neat polymer discs (see Appendix D, the Supporting Information for a table of swelling dimensions).

### Example 21: Ionic Conductivity of TPE GPE Composites

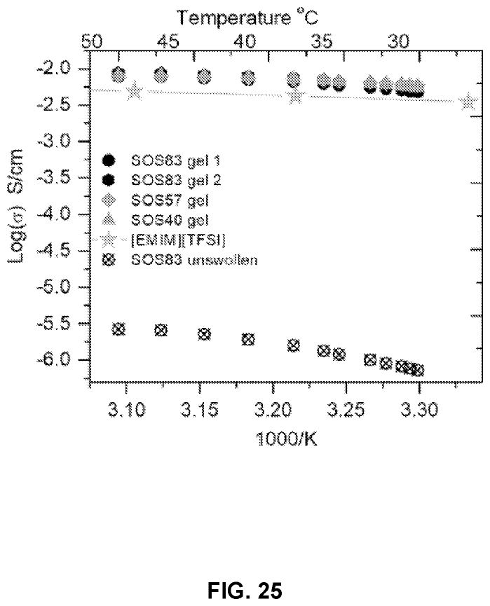

The ionic conductivities (shown in Table 3, FIG. 25) of the SOS ion gels (SOS83, SOS57, and SOS40), the neat ionic liquid, [EMIM][TFSI], and unswollen SOS83 discs were measured using electrochemical impedance spectroscopy (EIS) while under vacuum.

TABLE 3
Summary of swelling behavior and ionic conductivity of SOS ion gels and unswollen polymer

| | Amount of | | RTIL | conductivity (σ) | σ |
|---|---|---|---|---|---|
| | Triblock | Q range | loading | 1^{st} cycle | 2^{nd} cycle |
| | wt % in the | g RTIL | wt % in the | mS/cm at | mS/cm at |
| | neat blend | g SOS | gel | 50° C.-30° C. | 80° C.-30° C. |
| SOS83 gels | 83 | 8.7-10.7 | 89.7-91.4 | 7.73-4.75 (gel 1) | 11.6-4.21 (gel 1) |
| SOS83 neat | | n/a | n/a | 8.70-5.27 (gel 2) | 12.7-4.77 (gel 2) |
| | | | | 2.65 × 10⁻³-7.25 × 10⁻⁴ | 0.583-0.0167 |
| SOS57 gel | 57 | 12.5-16.2 | 92.6-94.2 | 8.28-5.43 | 9.98-5.29 |
| SOS40 gel | 40 | 14.5-21.1 | 93.5-95.5 | 7.89-5.87 | 9.78-5.06 |
| [EMIM][TFSI] | n/a | n/a | n/a | 4.85-3.47 | 6.34-3.47 |

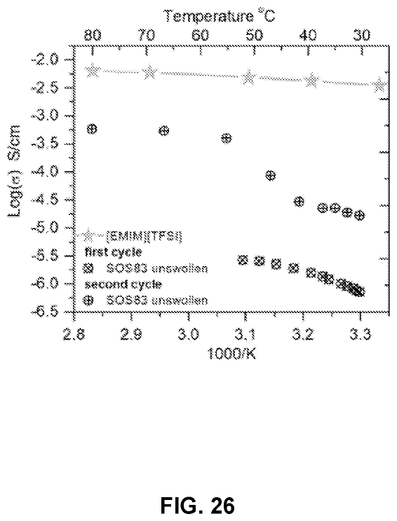

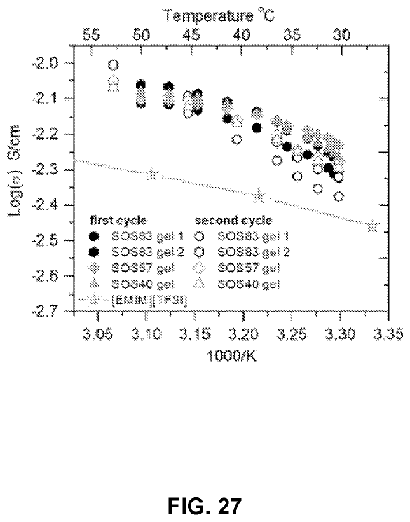

FIG. 25 shows the ionic conductivity of all samples as a function of temperature, measured between room temperature and 50° C. Ionic conductivity values for the neat ionic liquid, [EMIM][TFSI], are consistent with literature values (ca. 10⁻² S^{cm-1).16,17,29-32} Surprisingly, all SOS gels tested presented slightly higher ionic conductivity values than the neat RTIL at all temperatures, suggesting that the suspension of RTIL in the PEO matrix of the SOS gels seems to enhance its conductivity. This enhanced conductivity was, in part, due to some residual salt contamination in the SOS polymer associated with the anionic polymerization process, as the neat SOS83 polymer had a significantly higher conductivity (ca. 10⁻⁶ to 10⁻³ S cm⁻¹) than would be expected for pure PEO (10⁻⁹ to 10⁻⁷ S cm⁻¹).³³ This was found to be particularly true when the polymer was heated beyond the melting point of crystalline PEO domains (>55° C., see FIG. 26), granting mobility to both the polymer chains and the ions trapped within them. Interestingly, however, results of a recent conductivity study of salt-doped ionic liquids suggested that salt concentration had only a minor effect on the ionic conductivity of ionic liquid, and actually served to suppress the ionic conductivity of the neat RTIL slightly.³⁰ Additionally, GPE materials in the literature show ionic conductivities that are comparable to or lower than the neat electrolyte.^{11,12,15-18,34,35} These studies together suggest that there may be factors related to the molecular interactions between RTIL and PEO that are enhancing the conductivity of our gels over the neat ionic liquid, an effect that warrants further investigation. Certainly, the high RTIL loading (90 to 96 wt % RTIL) was a major contributor to the excellent conductivity of these SOS gels, and interestingly, the variation in RTIL loading among the three blends did not appear to have a substantial effect on conductivity performance. Mechanical softening of the gels (see FIG. 5.6) under extended (up to 80° C.) thermal annealing during a second cycle of testing did not affect the ionic conductivity significantly (see FIG. 26 and FIG. 27); conductivity values of the gels at 30° C. after the second cycle were only marginally lower than the initial cycle but were still above that of the neat RTIL.

### Example 22: General Evaluation of Mechanical Performance of the TPE GPE Composites

The range of mechanical performance of GPE materials is broad and exceedingly varied, however, when investigating potential GPEs prepared from physically crosslinked block copolymer systems that scope narrows considerably. Often, the mechanical performance is described by one advantageous mechanical property that the material has exhibited, such as high ultimate tensile strength or stretchability, while the tradeoffs in ionic conductivity or other aspects of mechanical performance (or both) as a direct result of achieving that favorable property are largely de-emphasized or not demonstrated at all. Consequently, our goal for this study was to perform a comprehensive mechanical investigation that could highlight the full potential of our SOS ion gel materials, and perhaps physically crosslinked BCP materials in general. To probe the mechanical behavior of these gels, we performed uniaxial tensile and compression testing, as well as rheology, to glean information on mechanical strength, stiffness, stretchability, elasticity, toughness, cyclability, hysteresis, and viscoelastic response.

In recent years, with the advent of tougher, more mechanically advanced soft materials, particularly highly swollen TPE gel polymer electrolyte composites, investigation of fracture mechanics has become essential for adequately evaluating the suitability (i.e. fracture resistance, toughness, and failure mechanisms) of these materials in mechanically demanding environments.²⁷ In this chapter, we have included a preliminary fracture toughness study of our SOS ion gels, which is, to the best of our knowledge, the first report of fracture toughness of physically crosslinked BCP GPEs.

### Example 23: Uniaxial Tensile Testing of TPE GPE Electrolytes

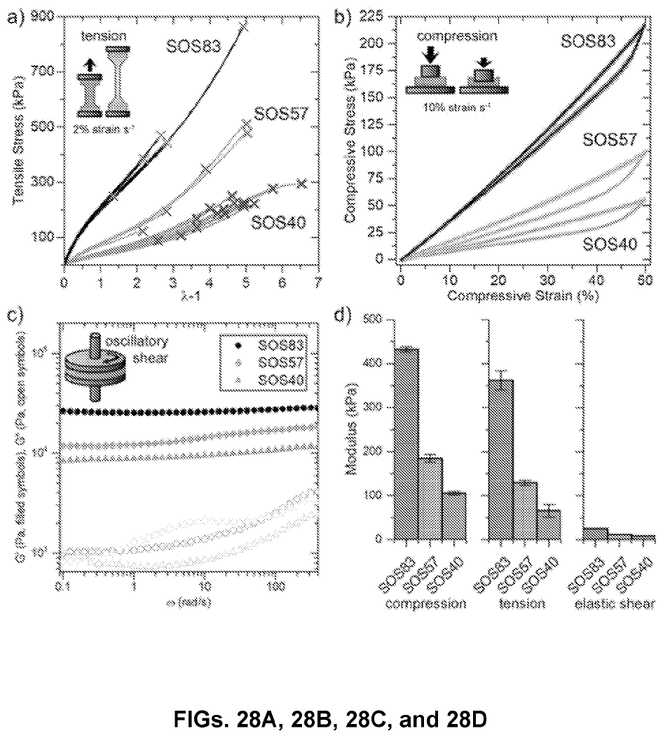

FIGS. 28A, 286, 28C, and 28D show the results of uniaxial tensile testing, unconfined compression testing, and dynamic frequency sweeps for SOS GPE elastomers and Table 3 table 5.3 provides a summary of these results.

TABLE 3
Summary of mechanical properties of SOS ion gels

| | Young's | | Ultimate | | | | |
|---|---|---|---|---|---|---|---|
| | Modulus | Elongation | Tensile | Bulk | Compressive | Fracture Toughness | |
| | (tensile) | at Break | Strength | Toughness | Modulus | f_{c} | fᵢₙ |
| | kPa | λ−1 | kPa | kJ/m³ | kPa | J/m² | |
| SOS83 | 391 | 4.9 | 863 | 2026 | 436 | — | — |
| | (maximum) | | | | | | |
| | 362 ± 22 | 2.8 ± 1.3 | 482 ± 229 | 817 ± 706 | 432 ± 5 | | |
| | (mean) | | | | | | |
| SOS57 | 134 | 5.1 | 511 | 1079 | 194 | — | — |
| | 129 ± 6 | 3.8 ± 1.3 | 331 ± 171 | 627 ± 427 | 184 ± 9 | | |
| SOS40 | 91 | 6.5 | 294 | 883 | 109 | 5233 | 208 |
| | 66 ± 13 | 4.5 ± 1.0 | 200. ± 56 | 411 ± 182 | 106 ± 4 | 2772 ± 1785 | 191 ± 15 |

FIG. 28B shows the SS83 gels exhibited excellent ultimate tensile strength (UTS) and stretchability, with one sample reaching nearly 0.9 MPa at 500% strain. This is remarkably high UTS for a homogenous, single network material that is comprised of only 10% polymer by weight and is physically crosslinked. SS57 blends reached UTS values upwards of 500 kPa at −500% strain, and even the softest SOS40 blends were able to reach −250 kPa with exceptional extensibilities up to nearly 8× without yielding, with most of the specimens reaching 400 to 600% strains. While some of our tensile specimens failed at lower elongations, none failed below 100% strain, and all but one above 200%. It is believed that those were likely a product of microscopic defects in the material due to imperfect laboratory processing techniques and equipment, to which uniaxial tensile testing can be very sensitive. The vast majority of these SOS gels were stretched to failure well past 300% strain, with even the “worst” samples being stretchable far beyond typical brittle or weak GPE materials.

TABLE 4
Summary of mechanical properties of SOS ion gels

| | Young's | | Ultimate | | | | |
|---|---|---|---|---|---|---|---|
| | Modulus | Elongation | Tensile | Bulk | Compressive | Fracture Toughness | |
| | (tensile) | at Break | Strength | Toughness | Modulus | f_{c} | fᵢₙ |
| | kPa | λ−1 | kPa | kJ/m³ | kPa | J/m² | |
| SOS83 | 391 | 4.9 | 863 | 2026 | 436 | — | — |
| | (maximum) | | | | | | |
| | 362 ± 22 | 2.8 ± 1.3 | 482 ± 229 | 817 ± 706 | 432 ± 5 | | |
| | (mean) | | | | | | |
| SOS57 | 134 | 5.1 | 511 | 1079 | 194 | — | — |
| | 129 ± 6 | 3.8 ± 1.3 | 331 ± 171 | 627 ± 427 | 184 ± 9 | | |
| SOS40 | 91 | 6.5 | 294 | 883 | 109 | 5233 | 208 |
| | 66 ± 13 | 4.5 ± 1.0 | 200. ± 56 | 411 ± 182 | 106 ± 4 | 2772 ± 1785 | 191 ± 15 |

It is important to note that the tensile strain-to-break data of SOS40 originates from two different blends, one prepared by precipitation and the other by freeze drying. The amount of SOS triblock and mechanical results for both blends were virtually indistinguishable and therefore have been pooled into results for just “SOS40.” All further mechanical results reported were performed on the freeze-dried SOS40 blend only because of the limited amounts of the precipitated blend. It is also important to note that the mechanical performance of the material is irrespective of the method in which the blend is prepared because the phase separated network structure is formed during subsequent thermal processing of those blends.

These tensile strain-to-break results translate to achievable bulk toughness values of over 2 MJ m⁻³ for SOS83, 1 MJ m⁻³ for SOS57, and nearly 900 kJ m⁻³ for SOS40, exceeding any tensile extension behavior observed in SOS gel materials to date^{17,22,23,25} and without the need for irreversible chemical crosslinking¹⁷ or a sacrificial secondary network to strengthen them as is the case for highly stretchable double network gels.^{36,37} FIGS. 28A, 28B, and 28C illustrate the mechanical advantage of our SOS gels over a traditional covalently crosslinked system. While the vitrified PS cores provide mechanical integrity as fixed network junction points, the solubilized PEO chains forming topologically fixed and dynamic entanglements contribute to the modulus of the gel in a manner similar to additional chemical crosslinks, but with the ability to redistribute stress concentrations by sliding past one another when extended or compressed. The absence of “fixed” strand lengths between the topological entanglements greatly increases the maximal extension and toughness of the gel and is reminiscent of the slide-ring systems developed by Ito and coworkers.³⁸

### Example 24: Unconfined Compression of TPE GPE Composites

By simply modifying the amount of SOS triblock in the blend, we can control the number of chains that can form those topological “crosslinks,” which in turn directly affects the swelling ratio, modulus, and strength of the material while only impacting potential extensibility to a minor degree. We have demonstrated excellent control of modulus—particularly compressive modulus—with typical standard deviations within each blend and test type of less than 5%. The cyclical unconfined compression results (FIG. 28B) show the outstanding compressibility, elasticity, and negligible hysteresis of these materials at all levels of SOS triblock content. A small amount of single-cycle hysteresis is expected, even in an ideal swollen elastomer system, due to forced redistribution of liquid within the gel as it is compressed. This is particularly pronounced at the fast strain rates (10% s⁻¹) used in this study. Upon decompression, mass transfer limitations within the highly entangled PEO matrix cause a very slight delay in the gel's return to its original conformation, however, the deformation is not permanent, and the gels fully elastically recover before the next compression cycle begins. Additionally, the modulus and compressive strength remained constant over all 10 cycles for each gel, which suggests that not only is the recovery of this material exceptionally rapid (<1 s) but that the network does not sustain any permanent damage from repeated compressive loading. Interestingly, the compressive hysteresis in the SOS83 ion gels studied in this chapter was appreciably smaller than the hysteresis observed in SOS83 (11-SOS) in chapter 4, likely due to the increased viscosity, molecular weight, and ionic interactions, which may prevent the RTIL from redistributing throughout the gel as quickly as water does upon decompression.

### Example 25: Dynamic Rheological Analysis of the TPE GPE Composites

Rheological frequency (ω) sweeps further confirmed the elastic nature of gels formed from all three blends. In FIG. 28C, the storage modulus (G′) exhibits plateau behavior typical of elastomeric materials and significantly exceeds the loss modulus (G″) for each gel at all frequencies—by an order of magnitude or more for most values of ω—indicating a stable, solid-like polymer network structure. The upward trend of the loss modulus at higher frequencies, however, suggests that while the polymer network remained solid and elastic at higher strain rates, the viscous contributions to the gels' complex modulus and are not completely frequency independent.

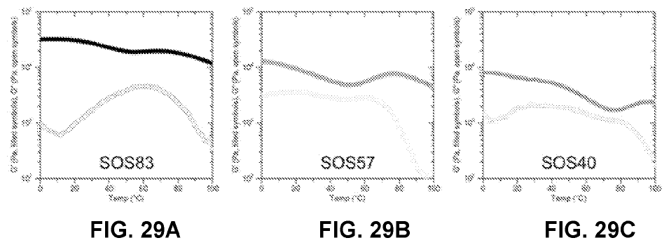

With increasing temperature, we observed a decrease in storage modulus during dynamic temperature ramps for all three blends (FIG. 29). Between 0 and 60° C., the [EMIM][TFSI] experiences an order of magnitude drop in viscosity,¹ from ˜100 mPa·s to ˜10 mPa·s and could be a contributor to the observed change in modulus over the temperature range tested. At some point beyond 50° C. the PS cores became viscous and the gels softened irreversibly, however, the storage modulus remained higher than the loss modulus over the full temperature range and the gels retained their shape when removed from the rheometer at room temperature. At equilibrium swelling, the PEO chains in the gels are already under stress such that once the PS domains soften above their Tg, PS chain rearrangement leads to a reduced modulus without disruption of the network. The solubility of PS in [EMIM][TFSI] is extremely poor up to and well past 100° C., so it is unlikely that plasticization of the PS spheres by the RTIL at elevated temperatures is responsible for softening the gels.³⁹

### Example 26: Cyclability of the TPE GPE Composites Under Tensile Strain

Lewis and coworkers previously demonstrated the excellent compressive fatigue resistance of our SOS TPE gel polymer electrolyte composites in which the gels were subject to fast, low strain (1 Hz cycles, 12% strain s⁻¹) but extraordinarily high cycle (up to 500k continuous) measurements to mimic repeated strains of tissue in a biomechanical environment such as the knee meniscus with nearly complete recovery of the modulus after short rest periods.²³ While it was unfeasible to repeat these extended compressive fatigue experiments for the ion gels explored in this study, based on the 10-cycle compression experiments and the similar chemical and morphological structure, we have reason to believe that swapping out water for RTIL as the liquid component of the gel should not significantly impact the compressive fatigue resistance of the network.

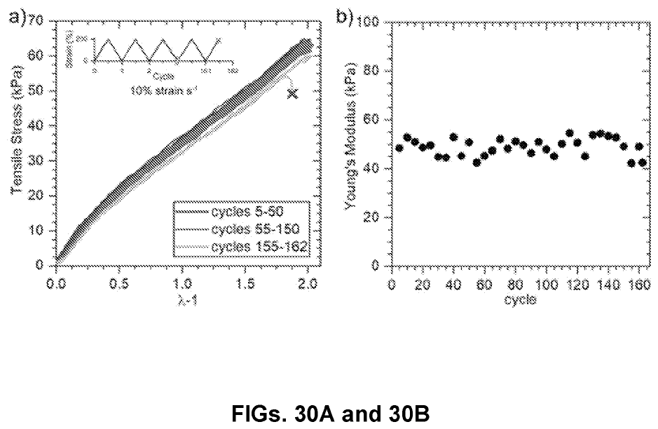

For applications in soft, flexible, and foldable electronics, it crucial to observe the cyclability of our SOS ion gels under repeated tensile strain, however true high-cycle “fatigue” testing was not within the scope of the study due to instrumentation limitations. Therefore, a test was chosen to test a sample at a high strain amplitude and rapid strain rate to demonstrate cyclability of the material under relatively extreme conditions over the course of a few hundred cycles, instead of many thousands. An SOS40 ion gel was pulled repeatedly to 200% strain (at 10% s⁻¹) until failure, and this data is shown in FIG. 30A. 200% strain was chosen as the highest strain that was still comfortably below the failure strength range for SOS40 gels, based on the tensile strain-to-break data. This gel was able to be stretched to 3× its initial length over the course of 161 cycles before failure at cycle 162, likely due to a defect in the material that caused a propagating crack. Importantly, over the course of those cycles, the initial (Young's) modulus of the material did not decay appreciably (shown in FIG. 30B), which indicates that without any flaws in the gel to initiate a tear, the material could be cycled under tensile strain for significantly longer since the elastic network is not being damaged with repeated loading. The elastic modulus at strains approaching 200% did decay slightly during the last ˜10 cycles or so, presumably when the crack began to propagate in the gel. The gel also showed excellent elasticity throughout the experiment, with no detectable hysteresis during each unloading cycle, even when the gel was close to failure.

### Example 27: Fracture Mechanics and Fracture Toughness of TPE GPE Composites

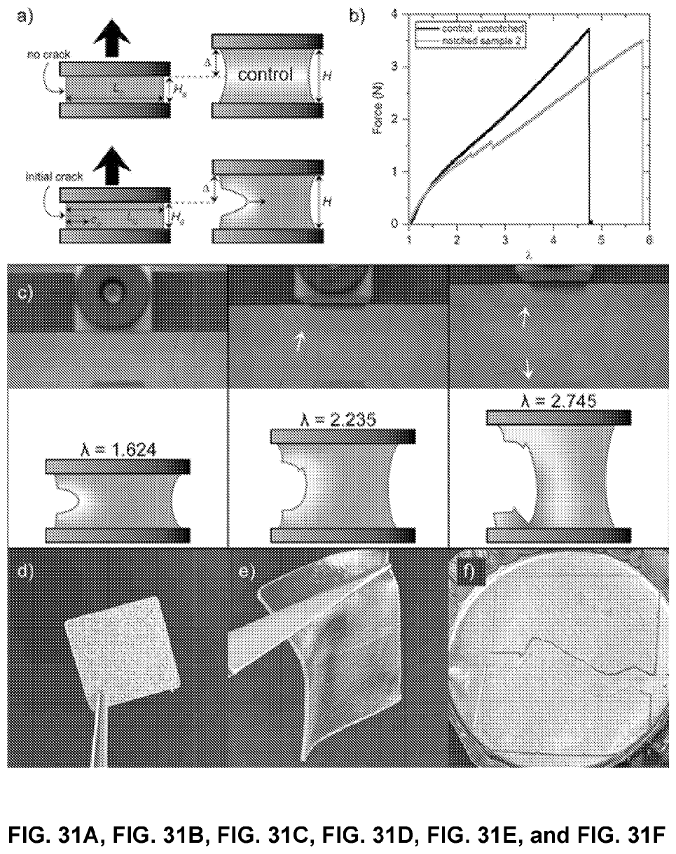

In “pure shear” fracture testing,^{26,27} the fracture toughness Gc, defined as the energy required to advance the crack by a unit area, can be evaluated (FIG. 31A). For isotropic elastic gels, the crack is expected to propagate horizontally (i.e., perpendicular to the applied tension) in an unstable manner.²⁷ The onset of unstable crack propagation should correspond to the peak force experienced by precut samples, based on which we determine the critical stretch ratio.

Using the approach above, it was found a large range for Gc, i.e., 1053 to 5233 J m⁻² among three SOS40 gels tested. In particular, the largest Gc (5233 J m⁻²) was for a notched sample of which the critical stretch (5.86) exceeded that of the unnotched sample (4.74) (FIG. 31B). This is surprising since intuitively the initial crack in the notched sample should reduce the critical stretch. As a result, we had to fit the force-displacement curve of the unnotched sample and extrapolate it beyond the critical stretch of failure. To resolve this apparent paradox, we examined images of the crack propagation process and discovered a crack branching behavior in all three notched gels tested; after the initial crack started to propagate, secondary sideways cracks emerged around the blunted tip of the primary crack (FIG. 31C). Formation of the sideways cracks caused local unloading as evidenced by the teeth-like profile in FIG. 31B, where we observed slight force drops as the propagating crack branched. This crack branching behavior—which was also observed in the two other notched samples—can arrest the primary crack or deflect its propagation direction, which eventually results in a tortuous crack path (FIG. 31F).

The physical mechanism behind crack branching is not known for these particular materials. Crack branching or deflection often occurs near filler/matrix interfaces in composite materials. For example, crack branching was observed in elastomeric composites with stiff and soft composites⁴⁰ and was exploited as a strategy to enhance fracture toughness and fatigue resistance.^{41,42} However, our ion gels have an amorphous network and are macroscopically homogeneous. In a recent work, crack branching was also found in a homogenous silicone elastomer,⁴³ which was attributed to the structural rearrangement of polymer chains near the crack tip due to high stretch. Whether this is true for our ion gels needs to be investigated by molecular characterizations such as X-ray scattering, which will be pursued in future works.

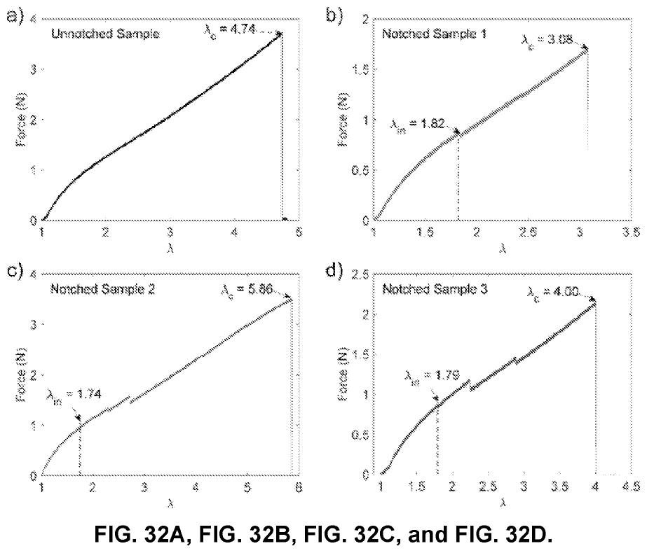

Regardless of the physical origin, crack branching serves as a mechanism to enhance the fracture toughness in our SOS ion gels without requiring sacrificial bonds.⁴⁴ To illustrate this point, we identified the stretch ratio, #$, at the onset of crack propagation (see FIG. 32A, FIG. 32B, FIG. 32C, and FIG. 32D and Table 5 and calculated the corresponding fracture toughness, G,n, for initiating crack propagation.

TABLE 5
Summary of the fracture toughness of SOS40 gels at the initiation of
crack propagation (┌in) and at the point of failure (┌c)

| | Fracture toughness I′ | Ultimate toughness |
|---|---|---|
| Notched | 207.80 | 1052.59 |
| Notched | 171.97 | 5232.5* |
| Notched | 193.94 | 2031.46 |

It was found that G,n fell into a much narrower range (172-208 J m⁻²). For a typical homogenous gel material that does not exhibit crack branching, at the onset of crack propagation the crack would propagate in the same direction as the initial crack until failure, and indeed, typical Gc values for physically crosslinked block copolymer and traditional TPE gel polymer electrolyte composites in the literature are very low (<100 J^{m-2).45-47} In contrast, the Gc of our gels is orders of magnitude higher (1000 to 5000 J m⁻²) and its variation from sample to sample is much larger. This significant difference in effective toughness is most certainly due to the crack branching effect, which causes stable crack propagation and delays ultimate failure. We can attribute the large variation in Gc to the stochastic nature of crack branching, but despite that, all three gels performed well into the range of “tough” materials like DN gels, composites, or liquid-free elastomers.^{36,40,44}

### REFERENCES

The entirety of the disclosures of the following references, and any other reference disclosed herein, are incorporated herein by reference.
1. P. Fröba, H. Kremer and A. Leipertz, J. Phys. Chem. B, 2008, 112, 12420-12430, DOI:10.1021/jp804319a
2. D. Zhou, D. Shanmukaraj, A. Tkacheva, M. Armand and G. Wang, Chem, 2019, 5, 2326-2352, DOI:10.1016/j.chempr.2019.05.009
3. Y. M. Kim, W. Y. Choi, J. H. Kwon, J. K. Lee and H. C. Moon, Chem. Mater., 2021, 33, 2683-2705, DOI:10.1021/acs.chemmater.1c00330
4. M. Ha, W. Zhang, D. Braga, M. J. Renn, C. H. Kim and C. D. Frisbie, ACS Appl. Mater. Interfaces, 2013, 5, 13198-13206, DOI:10.1021/am404204q
5. D. G. Mackanic, M. Kao and Z. Bao, Adv. Energy Mater., 2020, 10, 2001424, DOI:10.1002/aenm.202001424
6. H. C. Moon, T. P. Lodge and C. D. Frisbie, Chem. Mater., 2014, 26, 5358-5364, DOI:10.1021/cm502491n
7. H. C. Moon, T. P. Lodge and C. D. Frisbie, J. Am. Chem. Soc., 2014, 136, 3705-3712, DOI:10.1021/ja5002899
8. H. Hwang, S. Y. Park, J. K. Kim, Y. M. Kim and H. C. Moon, ACS Appl. Mater. Interfaces, 2019, 11, 4399-4407, DO1:10.1021/acsami.8b20004
9. J. Bruns and J. F. Stoddart, Acc. Chem. Res., 2014, 47, 2186-2199, DOI:10.1021/ar500138u
10. Kaneko, J. P. Gong and Y. Osada, J. Mater. Chem., 2002, 12, 2169-2177, DOI:10.1039/B109981G
11. W. Y. Choi, Y. M. Kim, H. Ahn and H. C. Moon, J. Mater. Chem. C, 2020, 8, 17045-17053, DOI:10.1039/DOTC04521G
12. Y. M. Kim and H. C. Moon, Adv. Funct. Mater., 2020, 30, 1907290, DOI:10.1002/adfm.201907290
13. K. Xu, Chem. Rev., 2014, 114, 11503-11618
14. S. Zhang, K. H. Lee, J. Sun, C. D. Frisbie and T. P. Lodge, Macromolecules, 2011, 44, 8981-8989, DOI:10.1021/ma201356j
15. T. Nguyen, Y. Zhu, X. Chen, G. A. Sotzing, S. Granados-Focil and R. M. Kasi, J. Mater. Chem. C, 2015, 3, 399-408, DOI:10.1039/c4tc01702a
16. C. Wei, M. Chen, D. Liu, W. Zhou, M. Khan, X. Wu, N. Huang and L. Li, Polym. Chem., 2015, 6, 4067-4070, DOI:10.1039/c5py00366k
17. Y. Gu, S. Zhang, L. Martinetti, K. H. Lee, L. D. McIntosh, C. D. Frisbie and T. P. Lodge, J. Am. Chem. Soc., 2013, 135, 9652-9655, DOI:10.1021/ja4051394
18. H. Mizuno, K. Hashimoto, R. Tamate, H. Kokubo, K. Ueno, X. Li and M. Watanabe, Polymer (Guildf)., 2020, 206, 122849, DOI:10.1016/j.polymer.2020.122849
19. C. Guo and T. S. Bailey, Soft Matter, 2010, 6, 4807-4818, DOI:10.1039/cOsm00139b
20. P. G. Santangelo and C. M. Roland, Macromolecules, 1998, 31, 4581-4585, DOI:10.1021/ma971823k
21. C. Guo and T. S. Bailey, Soft Matter, 2015, 11, 7345-7355, DOI:10.1039/c5sm00122f
22. C. Guo, J. T. Lewis, V. F. Scalfani, M. M. Schwartz and T. S. Bailey, Chem. Mater., 2016, 28, 1678-1690, DOI:10.1021/acs.chemmater.5b04431
23. J. T. Lewis, K. M. Fischenich, T. L. Haut Donahue and T. S. Bailey, ACS Biomater. Sci. Eng., 2018, 4, 3854-3863, DOI:10.1021/acsbiomaterials.8b00929
24. A. Finotello, J. E. Bara, D. Camper and R. D. Noble, Ind. Eng. Chem. Res., 2008, 47, 3453-3459, DOI:10.1021/ie0704142
25. B. Wijayasekara, M. G. Cowan, J. T. Lewis, D. L. Gin, R. D. Noble and T. S. Bailey, J. Memb. Sci., 2016, 511, 170-179, DOI:10.1016/j.memsci.2016.03.045
26. R. S. Rivlin and A. G. Thomas, J. Polym. Sci., 1953, 10, 291-318, DOI:10.1002/POL.1953.120100303
27. R. Long and C. Y. Hui, Soft Matter, 2016, 12, 8069-8086
28. N. A. Huq and T. S. Bailey, Macromolecules, 2018, 7734-7744
29. M. L. Hoarfrost and R. A. Segalman, Macromolecules, 2011, 44, 5281-5288, DOI:10.1021/ma200060g
30. D. Monti, E. Jónsson, M. R. Palacin and P. Johansson, J. Power Sources, 2014, 245, 630-636, DOI:10.1016/j.jpowsour.2013.06.153
31. M. G. Cowan, A. M. Lopez, M. Masuda, Y. Kohno, W. M. McDanel, R. D. Noble and D. L. Gin, Macromol. Rapid Commun., 2016, 37, 1150-1154, DOI:10.1002/MARC.201600029
32. H. Tokuda, K. Hayamizu, K. Ishii, M. A. B. H. Susan and M. Watanabe, J. Phys. Chem. B, 2005, 109, 6103-6110, DOI:10.1021/jp044626d
33. V. M. Mohan, P. B. Bhargav, V. Raja, A. K. Sharma and V. V. R. Narasimha Rao, Soft Mater., 2007, 5, 33-46, DOI:10.1080/15394450701405291
34. Y. He, P. G. Boswell, P. Bühlmann and T. P. Lodge, J. Phys. Chem. B, 2007, 111, 4645-4652, DOI:10.1021/jp064574n
35. B. Tang, S. P. White, C. D. Frisbie and T. P. Lodge, Macromolecules, 2015, 48, 4942-4950, DOI:10.1021/acs.macromol.5b00882
36. J. Y. Sun, X. Zhao, W. R. K. Illeperuma, O. Chaudhuri, K. H. Oh, D. J. Mooney, J. J. Vlassak and Z. Suo, Nature, 2012, 489, 133-136, DOI:10.1038/nature11409
37. H. Wu, Y. Cao, H. Su and C. Wang, Angew. Chemie Int. Ed., 2018, 57, 1361-1365, DOI:10.1002/anie.201709774
38. K. Kato and K. Ito, React. Funct. Polym., 2013, 73, 405-412, DOI:10.1016/j.reactfunctpolym.2012.04.021
39. X. Chen, C. Zhou, S.-J. Chen, G. S. W. Craig, P. Rincon-Delgadillo, T. Dazai, K. Miyagi, T. Maehashi, A. Yamazaki, R. Gronheid, M. P. Stoykovich and P. F. Nealey, ACS Appl. Mater. Interfaces, 2018, 10, 16747-16759, DOI:10.1021/acsami.8b02990
40. Z. Wang, C. Xiang, X. Yao, P. Le Floch, J. Mendez and Z. Suo, Proc. Natl. Acad. Sci. U.S.A., 2019, 116, 5967-5972, DOI:10.1073/pnas.1821420116
41. C. Xiang, Z. Wang, C. Yang, X. Yao, Y. Wang and Z. Suo, Mater. Today, 2020, 34, 7-16, DOI:10.1016/j.mattod.2019.08.009
42. C. Li, H. Yang, Z. Suo and J. Tang, J. Mech. Phys. Solids, 2020, 134, 103751, DOI:10.1016/j.jmps.2019.103751
43. S. Lee and M. Pharr, Proc. Natl. Acad. Sci. U.S.A., 2019, 116, 9251-9256, DOI:10.1073/pnas.1820424116
44. J. P. Gong, Soft Matter, 2010, 6, 2583, DOI:10.1039/b924290b
45. K. J. Henderson, T. C. Zhou, K. J. Otim and K. R. Shull, Macromolecules, 2010, 43, 6193-6201, DOI:10.1021/ma100963m
46. M. E. Seitz, D. Martina, T. Baumberger, V. R. Krishnan, C.-Y. Hui and K. R. Shull, Soft Matter, 2009, 5, 447-456, DOI:10.1039/B810041A
47. D. Bonn, H. Kellay, M. Prochnow, K. Ben-Djemiaa and J. Meunier, Science (80-.)., 1998, 280, 265-267, DOI:10.1126/science.280.5361.265.

1. A thermoplastic elastomer (TPE) composite comprising a first styrenic thermoplastic elastomer and a second styrenic thermoplastic elastomer, wherein the first and second styrenic thermoplastic elastomers each independently comprise at least one non-hydrogenated or hydrogenated styrene block.

2. The TPE composite of claim 1, wherein the first styrenic thermoplastic elastomer comprises at least one block copolymer comprising at least one non-styrenic hydrophilic block, and the second styrenic thermoplastic elastomers comprises at least one block copolymer comprising at least one non-styrenic hydrophobic block.

3. The TPE composite of claim 1, wherein the first styrenic thermoplastic elastomer comprises at least one block copolymer comprising at least one non-styrenic hydrophilic block, and the second styrenic thermoplastic elastomer comprises at least one block copolymer comprising at least one non-styrenic hydrophilic block.

4. The TPE composite of claim 1, wherein the first styrenic thermoplastic elastomer comprises at least one block copolymer comprising at least one non-styrenic hydrophobic block, and the second styrenic thermoplastic elastomer comprises at least one block copolymer comprising at least one non-styrenic hydrophobic block.

5. The TPE composite of claim 1, wherein each of the first styrenic thermoplastic elastomer and/or the second styrenic thermoplastic elastomer comprises diblock copolymers, triblock copolymers, tetrablock copolymers, pentablock copolymers, or any combination thereof.

6. The TPE composite of claim 2, wherein the least one hydrophilic block comprises at least one polyalkylene oxide block.

7. The TPE composite of claim 6, wherein the at least one polyalkylene oxide block comprises a polyethylene oxide block.

8. The TPE composite of claim 2, wherein the at least one hydrophobic block comprises at least one hydrophobic non-glassy block.

9. The TPE composite of claim 8, wherein the at least one hydrophobic non-glassy block comprises a polydiene, a substituted polydiene, a hydrogenated polydiene, a substituted hydrogenated polydiene, a polysiloxane, a substituted polysiloxane, or any combination thereof.

10. The TPE composite of claim 1, wherein the ratio of the first styrenic thermoplastic elastomer to the second styrenic thermoplastic elastomer; is 1:99 to 99:1.

11. The TPE composite of claim 10, wherein the ratio of the first styrenic thermoplastic elastomer to the second styrenic thermoplastic elastomers is 1:4 to 4:1.

12. The TPE composite of claim 1, wherein the first styrenic thermoplastic elastomer and/or the second styrenic elastomer comprising at least one non-styrenic hydrophilic block comprises a polystyrene-polyethylene oxide-polystyrene triblock copolymer (SOS).

13. The TPE composite of claim 1, wherein the first styrenic thermoplastic elastomer and/or second styrenic thermoplastic elastomer comprising at least one non-styrenic hydrophobic block comprises at least one non-styrenic hydrophobic block selected from a polystyrene-polybutadiene-polystyrene triblock copolymer (SBS), a substituted SBS triblock copolymer, a polystyrene-polyisoprene-polystyrene triblock copolymer (SIS), a substituted SIS triblock copolymer, a polystyrene-poly(ethylene butylene)-polystyrene triblock copolymer (SEBS), a substituted SEBS triblock copolymer, a polycyclohexylethylene-poly(ethylene butylene)-polycyclohexylethylene triblock copolymer (PEBP), a substituted PEBP triblock copolymer, a polystyrene-polysiloxane-polystyrene triblock copolymer (SDS), a substituted SDS triblock copolymer, or any combination thereof.

14. The TPE composite of claim 1, wherein the at least two styrenic thermoplastic elastomers comprise SOS and SEBS.

15. The TPE composite of claim 14, wherein the ratio of SOS to SEBS is 1:19 to 19:1.

16. A method for preparing a thermoplastic elastomer (TPE) composite, comprising:
(a) contacting the at least two thermoplastic elastomers of claims 1-15 to form a TPE dry blend;
(b) heating the TPE dry blend to form a TPE composite melt; and
(c) allowing the TPE composite melt to attain ambient temperature to form an TPE composite of the at least two thermoplastic elastomers.

17. (canceled)

18. The method of claim 16, wherein the TPE dry blend is formed by heating the at least two thermoplastic elastomers between about 80° C. and 320° C.

19. (canceled)

20. (canceled)

21. The method of claim 169, wherein step (b), heating the TPE dry blend, further comprises mechanical mixing, compounding, or extrusion.

22. The method of claim 21, wherein step (b), heating the TPE dry blend, comprises mechanical mixing with a screw or mixing speed between about 50 rpm and 250 rpm.

23. The method of claim 16, wherein the TPE dry blend has a microstructure characterized by TPE domains of sizes of about 0.1 microns to about 10 microns, about 0.1 microns to about 20 microns, or about 0.1 microns to about 50 microns.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)
